(19) United States
(12) Patent Application Publication
(10) Pub. No.: US 2022/0126060 A1
(43) Pub. Date: Apr. 28, 2022

(54) REINFORCED CENTER LUMEN FOR STEERABLE DEVICE

(71) Applicant: CANON U.S.A., INC., MELVILLE, NY (US)

(72) Inventors: Benedict Shia, Needham, MA (US); Zachary Hamilton Haubert, Boston, MA (US); Christopher Charles Kaiser, Acton, MA (US); Takahisa Kato, Brookline, MA (US)

(21) Appl. No.: 17/508,753

(22) Filed: Oct. 22, 2021

Related U.S. Application Data
(60) Provisional application No. 63/104,935, filed on Oct. 23, 2020.
(60) Provisional application No. 63/112,931, filed on Nov. 12, 2020.

(51) Int. Cl. A61M 25/00; A61L 29/08; A61M 25/01

(52) U.S. Cl. CPC A61M 25/0045; A61M 25/005; A61M 2025/0047; A61L 29/085; A61M 25/0147; A61M 25/0009

(57) ABSTRACT

A catheter sheath comprises: a central lumen extrusion having an inner layer defining a central lumen, a reinforcing structure surrounding the inner layer, and an outer layer surrounding the reinforcing structure; a plurality of rings are arranged on the outer layer of the central lumen extrusion at a predetermined distance from each other in a distal section of the sheath; and an outer jacket encloses the rings and central lumen extrusion. The reinforcing structure includes one or more of a braid, a coil, and a laser-cut tube embedded between the inner layer and the outer layer; the central lumen extrusion is bonded with an adhesive or laser welded or pressure fit to one or more of the rings. The reinforcing structure is offset towards the inner or the outer surface; and the rings have chamfered or beveled or rounded inner edges.

### CROSS-REFERENCE TO RELATED APPLICATIONS

The present application claims priority to U.S. provisional application 63/112931 filed Nov. 12, 2020, and U.S. provisional application 63/104935 filed Oct. 23, 2020. The disclosures of the above-listed provisional applications are hereby incorporated by reference in their entirety for all purposes. Priority benefit is claimed under 35 U.S.C. § 119(e).

### BACKGROUND INFORMATION

### Field of Disclosure

The present disclosure relates to medical devices. More particularly, the disclosure exemplifies embodiments of a reinforced central lumen extrusion applicable for use in a tubular sheath of steerable medical devices, such as endoscopes or catheters.

### Description of Related Art

Medical devices configured for minimally invasive surgical (MIS) procedures include catheters and endoscopic probes. Some of these medical devices are guided through a disposable or limited-use flexible tubular body commonly referred to as a sleeve or sheath or introducer sheath. Some of these introducer sheaths or sleeves are robotically controlled. A robotically controlled catheter or endoscope has a catheter sheath with a steerable distal section and a non-steerable proximal section. The proximal section connects to an actuator unit via an electromechanical connector, and the distal section is sized to be introduced into a patient's anatomy through natural orifices or small surgical incisions. Similar catheter or endoscopes insertable into a patient can be operated manually by a user without automated or robotic control. In either case, one or more channels extend along a central lumen of the sheath to allow access for imaging devices (miniature cameras or optical fiber probes) and/or end effectors (biopsy tools or therapy probes), and/or for passing fluids (contrast agents, gas, or flushing solutions).

To reduce the exposure to fluids and minimize interaction with an instrument passing through the lumen, the sheath generally includes an inner liner which has its inner surface thereof configured to meet certain requirements such as lubricity, hydrophobicity, and flexibility. The inner liner may be an extrusion of thermoplastic or fluoropolymer materials, such as Pebax®, nylon, polyimide, high-density polyethylene (HDPE), Plexar®, urethane resin, or combinations thereof in the form of a thin-walled tube having an inner diameter sized according to design requirements. See, for example, U.S. Pat. Nos. 7,550,053, 7,553,387, 10,821,264, and pre-grant publications US 2009/0126862, the disclosures of which are incorporated by reference herein.

For robotically controlled sheaths, a plurality of driving wires or tendons extend along the wall of the sheath to allow the actuator unit to selectively manipulate (bend) the distal section of the sheath. In some designs, the distal section of the sheath has a plurality of bendable segments which include rings made of biocompatible polymer, such as polytetraflouroethylene (PTFE) or polyethylene (PE). These rings are bonded to the outer surface of the inner liner. The driving wires or tendons, which are typically made of metallic material such as nickel-titanium (NiTi) alloy (nitinol) or stainless steel or other similar metals, are guided through thru-holes (secondary lumens) provided in wall of the rings. This type of steerable medical devices, used for medical examination or treatment of internal body structures, are described in numerous patent-publications including, for example, pre-grant publication US 2016/0067450, international publication WO/2020/092097, and U.S. Pat. Nos.: 8,365,633, 9,144,370 and 10,687,694 the disclosures of which are hereby incorporated by reference in their entirety.

During use, as the sheath structure is bent with tight curvatures in a tortuous anatomy of a patient, the gap between the rings increases on the outer radius of the bend and reduces on the inner radius of the bend. This causes the inner liner to stretch in the outer radius of the bend and to crimp and create ridges on the inner radius of the bend. Then, when an instrument is passed through the central lumen of the sheath, the instrument can deflect enough so that the tip of the instrument can catch on a ring and try to poke out of the sheath in the space between two rings. On the other hand, for some inserted tools, the tool tip can get stuck on “ridge” between guide rings. The foregoing issues can cause damage to the catheter and to the instrument or tool indented to pass therethrough, and since the instrument cannot be passed, the procedure for which the instrument was intended cannot be performed.

Accordingly, there is a need for improved steerable medical devices, in particular robotically steerable catheters or endoscopes having reduced overall diameter, which require a thinner more flexible, yet torsional resistant central lumen.

### SUMMARY OF EXEMPLARY EMBODIMENTS

According to at least one embodiment of the present disclosure, there is provided an apparatus comprising a catheter sheath with a reinforced central lumen extrusion. A catheter sheath extending lengthwise from a proximal end to a distal end along a sheath axis, the catheter sheath comprising: a central lumen extrusion having multiple layers including an inner layer defining a central lumen, a reinforcing structure surrounding the inner layer, and an outer layer surrounding the reinforcing structure in this order substantially concentric to the sheath axis; and a plurality of rings arranged on the outer layer of the central lumen extrusion, wherein the plurality of rings are arranged at a predetermined distance from each other in a direction from the distal end towards the proximal end, wherein the reinforcing structure of the central lumen extrusion includes one or more of a braid structure, a coil structure, and a laser-cut tube structure embedded between the inner layer and the outer layer, and wherein the central lumen extrusion is bonded with an adhesive or laser welded or pressure fit to one or more of the plurality of rings.

According to one embodiment, the catheter sheath comprises: an elongated tubular body having a proximal end and a distal end and defining a central lumen extending the length of the tubular body along a sheath axis, the tubular body includes a steerable section formed of guide rings disposed collectively in a lengthwise direction of the tubular body, the guide rings are spaced a predetermined distance from each other to create a gap therebetwen. A central lumen extrusion having an inner surface, a reinforcing structure, and an outer surface arranged in this order substantially concentric to the central lumen between the tubular body and the central lumen; the reinforcing structure includes one or more of a braid structure, a coil structure, and a laser-cut tube structure embedded between the inner surface and the outer surface. The reinforcing structure is offset towards the inner or outer surface.

According to one embodiment, the steerable sheath comprises: an elongated tubular body having a proximal end and a distal end and defining a central lumen extending the length of the tubular body, wherein the tubular body includes a steerable section formed of guide rings disposed collectively in a lengthwise direction of the tubular body, wherein the guide rings are spaced a predetermined distance from each other to create a gap between each pair of consecutive guide rings, wherein the guide rings include wire conduits arranged substantially parallel to and equidistant from the central lumen; at least one control wire slideably arranged in each wire conduit, a distal end of the at least one control wire being attached to the steerable section of the tubular body and a proximal end of the at least one control wire configured to be mechanically connected to an actuator unit; and a central lumen extrusion having an inner surface, a reinforcing structure, and an outer surface arranged in this order substantially concentric to the central lumen between the tubular body and the central lumen, wherein the reinforcing structure includes one or more of a braid structure, a coil structure, and a laser-cut tube structure embedded between the inner surface and the outer surface.

According to certain embodiments, the central lumen extrusion includes an inner layer and an outer layer concentric to each other, and the one or more of a braid reinforcing structure, a coil reinforcing structure, and a laser-cut tube reinforcing structure is enclosed between the inner layer and the outer layer of the central lumen extrusion.

According to certain embodiments, the inner layer and the outer layer are both made of elastomeric polymer material, and the inner layer includes, or is coated with, a lubricious material not included in the outer layer.

According to certain embodiments, the outer layer is made of thermoplastic elastomer (TPE) and the inner layer is made of thermoplastic polyurethane (TPU).

According to certain embodiments, a thickness of the inner layer is larger than a thickness of the outer layer. Alternatively, a thickness of the outer layer is larger than a thickness of the inner layer.

According to certain embodiments, a durometer of the inner layer is different from a durometer of the outer layer. For example, the durometer of the inner layer is higher than a durometer of the outer layer. Alternatively, the durometer of the inner layer is lower than a durometer of the outer layer.

According to certain embodiments, the coil reinforcing structure included in the central lumen extrusion is a first coil reinforcing structure made of a metallic wire and/or polymeric wire coiled in a first direction with respect to the lumen axis, and the outer jacket includes a second coil reinforcing structure made of a metallic wire and/or a polymeric wire coiled in a second direction with respect to the lumen axis, and the first direction is opposite to the second direction.

According to certain embodiments, the outer layer is made of elastomeric polymer combined with carbon black additive material, and the inner layer is made of elastomeric polymer combined or coated with a lubricious additive.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

FIG. 1A illustrates an example embodiment of a medical system 1000 including a steerable medical device 11 in an applicable medical environment thereof. FIG. 1B illustrates an example embodiment of the medical system 1000 in bock diagram form;

FIG. 2A and FIG. 2B illustrate structural details of a steerable catheter sheath 100 with a central lumen extrusion 200 and a plurality of guide rings arranged on the central lumen extrusion. FIG. 2C shows examples of guide rings having wire-guiding conduits for wires; FIG. 2D illustrate ring components having alternate structures or functions;

FIG. 3 illustrates an example embodiment of a central lumen extrusion 200 comprising a reinforcing structure made of braided fibers;

FIG. 4 illustrates an example embodiment of a central lumen extrusion 200 comprising a reinforcing structure made of coiled wire;

FIG. 5 illustrates an example embodiment of a central lumen extrusion 200 comprising a reinforcing structure made of laser-cut tube;

FIG. 6A shows an example embodiment of the central lumen extrusion 200. FIG. 6B shows a cross-sectional view of the reinforced central lumen extrusion 200, as seen along section B-B of FIG. 6A. FIG. 6C and FIG. 6D respectively show a cross-sectional view of the reinforced central lumen extrusion 200, as seen along section B-B of FIG. 6A, where the reinforcing structure 220 is offset with respect to the inner and outer layers;

FIG. 7 illustrates an example embodiment of a reinforced central lumen extrusion 200 where thin-walled rings 720 (reinforcing rings) are located on the outer surface of central lumen extrusion 200;

FIG. 8A illustrates an example embodiment of a reinforced central lumen extrusion 200. FIG. 8B shows a detailed view of an area B of FIG. 8A. FIG. 8C shows an example embodiment of a guide ring 120 with chamfered or beveled or rounded edges 825 in its inner surface thereof;

FIG. 9A and FIG. 9B illustrate an example embodiment of a reinforced central lumen extrusion with different gap distances between consecutive guide rings of a bending segment;

FIG. 10 illustrates an example embodiment of a steerable catheter sheath 100 having a reinforced central lumen extrusion 200 and an outer jacket 800; and

FIG. 11A shows a cross-sectional view of catheter sheath 100, as seen from a plane perpendicular to the lumen axis Ax. FIG. 11B shows a cross-sectional view of catheter sheath 100 wherein the outer jacket 800 includes a reinforcing structure similar to the reinforcing structure of the central lumen extrusion;

FIG. 12 illustrates an exemplary manufacturing process for making a catheter sheath having a central lumen extrusion with a reinforcing structure; and

FIG. 13 is a graph showing experimental results of bending a catheter sheath having a central lumen extrusion with a reinforcing structure.

### DETAILED DESCRIPTION

The following paragraphs describe certain explanatory embodiments of a robotic medical system configured to use a steerable medical device having a reinforced central lumen. Other embodiments may include alternatives, equivalents, and modifications. Additionally, the explanatory embodiments may include several features, and a particular feature may not be essential to some embodiments of the devices, systems, and methods that are described herein.

Throughout the figures, where possible, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

When a feature or element is herein referred to as being “on” another feature or element, such feature or element can be directly on the other feature or element or intervening features and/or elements may also be present. In contrast, when a feature or element is referred to as being “directly on” another feature or element, it is understood that there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, it is understood that there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms of order, such as first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, in particularly when used in a claim, the transitional phrase “consisting of” excludes any element, step, or component not specified in the claim. It is further noted that some claims or some features of a claim may be drafted to exclude any optional element; such claim or claims may use exclusive terminology as “solely,” “only” and the like in connection with the recitation of claim elements, or it may use of a “negative” limitation.

The term “about” or “approximately” as used herein means, for example, within 10%, within 5%, or less of a given quantity. In some embodiments, the term “about” may mean within measurement error or manufacturing tolerance. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical range, if recited herein, is intended to include the given limits and all sub-ranges subsumed therein. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g., substantially spherical, substantially perpendicular or parallel, substantially concentric, etc.) or a relative term (e.g. substantially similar, substantially the same, etc.).

The present disclosure generally relates to medical devices, and it exemplifies embodiments of a steerable catheter sheath for guiding a catheter and/or an optical probe which may be applicable to an imaging apparatus (e.g., an endoscope). The imaging apparatus may image using a miniature camera based on chip-on-tip (COT) technology, or may provide some other form of imaging such as spectrally encoded endoscopy (SEE) imaging technology (see, e.g., U.S. Pat. Nos. 10,288,868 and 10,261,223). In some embodiments, the imaging apparatus may include an optical coherence tomographic (OCT) apparatus, a spectroscopy apparatus, or a combination of such apparatuses (e.g., a multi-modality imaging probe).

The embodiments of the steerable instrument and portions thereof are described in terms of their positon/orientation in a three-dimensional space. As used herein, the term “position” refers to the location of an object or a portion of an object in the three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to a total six degrees of freedom); the term “shape” refers to a set of posture, positions, and/or orientations measured along the elongated body of the object. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The catheter may be solely an imaging apparatus or it may comprise tools for use in therapeutic or diagnostic procedures. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

Steerable medical instruments provide flexible access (e.g., access with one or more curves) to an intended lesion or other interior site while retaining torsional and longitudinal rigidity so that physicians can control the end effectors located at the distal end (the end closest to the interior site) by operating a proximal end (the end farthest from the interior site and closest to the physician) of the instrument. Some steerable medical instruments are robotized and use kinematic principles to actuate a bendable catheter sheath with driving wires actuated in push and pull directions to cause portions of the bendable body to bend. However, as noted above, to aces deep-seated lesions and other sites it is necessary to minimize an outer diameter (OD) and to maximize an inner diameter (ID) of the central lumen (or tool channel) of the catheter sheath . Therefore, some medical steerable instruments may comprise a sheath that has a minimal wall thickness which can be improved by a reinforced central lumen, as described in the present disclosure.

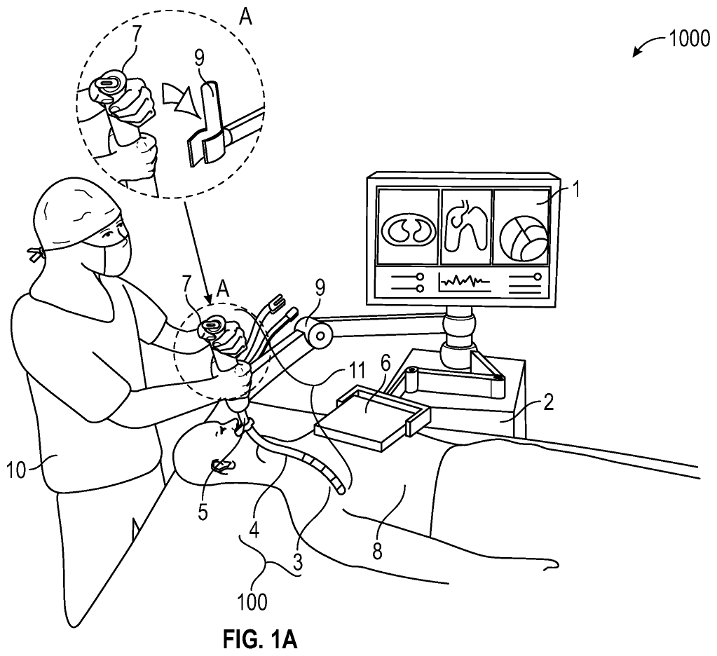

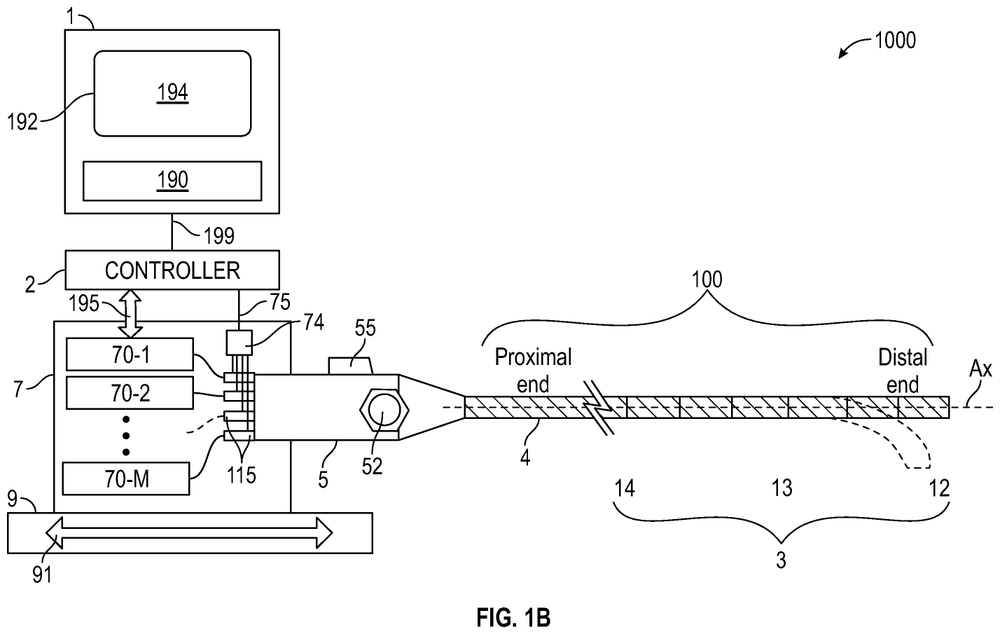

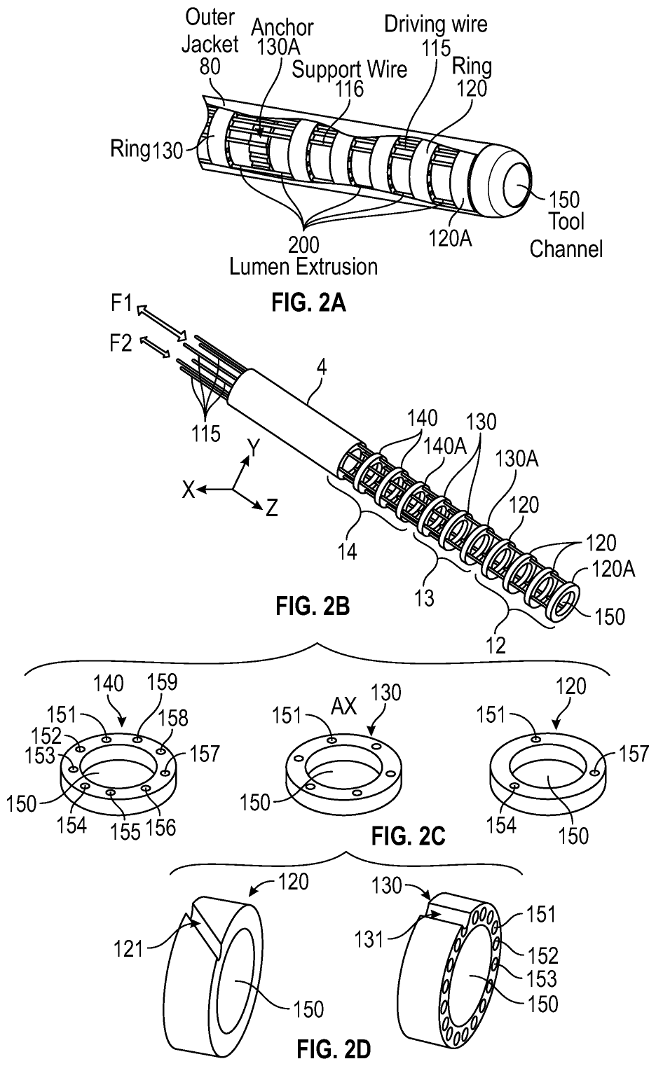

First, structural components of a robotic medical system 1000 comprising a bendable body 3 detachably attached to an actuation unit 7 via a connector assembly 5 will be described with reference to FIG. 1A, FIG. 1B, and FIG. 2A-2C. The robotic medical system 1000 can include a continuum or multi-segment robot configured to form a continuously curved geometry by actuating one or more bending sections of the bendable body 3. An example of a continuum robot is a snake-like endoscopic device, as described in applicant's previously published U.S. Pat. No. 9,144,370, and patent application publications US 2015/0088161, US 2018/0243900, US 2018/0311006 and US 2019/0015978, which are incorporated by reference herein for all purposes.

Most of these robotically steerable medical devices have polymer rings and metallic wires arranged around a central lumen so as to create a flexible backbone for snake-like articulation. Therefore, this type of steerable medical instruments is known as a snake or continuum robot. The snake-like continuum robot has a unique distal structure wherein the polymer rings are attached to the central lumen at a predetermined spacing to form a skeleton structure with specific bending properties. The central lumen can be a single lumen extrusion made of a low durometer material to reduce the forces required to bend the skeleton structure. The single lumen skeleton structure made of low durometer material can achieve a relatively tight bending radius. As the catheter sheath is bent into a curved shape, the gap between the rings increases on the outer radius and decreases on the inner radius of the curved structure. Then, when an instrument is passed through the curved central lumen, the low durometer of the central lumen extrusion can cause the wall of the lumen to deflect or contract enough so that the tip of the instrument can catch on the rings and become stuck. Specifically, the low durometer of the central lumen extrusion does not provide enough hoop strength to resist expansion or contraction (ovalization) of the central lumen in the radial direction. Therefore, there are instances where the central lumen cannot prevent deflection of the instrument and cannot avoid the instrument from catching on the rings of the skeleton structure. This situation can cause damage to the catheter sheath and/or to the instrument inserted through the central lumen, and it could also potentially hinder the safety of the patient.

According to the present disclosure, one or more embodiments are directed to a central lumen extrusion formed of an inner liner having an inner surface, a reinforcing structure, and an outer surface arranged in this order substantially concentric to a central lumen. The reinforcing structure includes one or more of a braid structure, a coil structure, and a laser-cut tube structure embedded between the inner surface and the outer surface of the inner liner. In at least some embodiments, the reinforcing structure is offset towards the inner surface or the outer surface. According to the present disclosure, one or more embodiments are directed to a catheter sheath comprising the central lumen extrusion and having a plurality of rings arranged lengthwise on the outer surface of the central lumen extrusion in a direction from the distal end towards the proximal end at a predetermined distance from each other. At least some rings have secondary lumens used as conduits for control wire or support wires to actuate the distal end of the catheter sheath. The outer surface of the central lumen extrusion and/or the inner surface of the rings are specifically designed to achieve tight bending of the catheter sheath in tortuous anatomies having curvatures greater than 90 degrees.

### FIG. 1A-1B: Robotic Medical System

A robotic medical system will be described by referring to FIG. 1A and FIG. 1B. FIG. 1A illustrates an example embodiment of a medical system 1000 in a medical environment such as an operating room (OR). The medical system 1000 makes use of a steerable instrument 11 (steerable medical device) to treat a patient 8 under interactive commands of a user (e.g., a physician) 10. The medical system 1000 includes at least a navigation system 1, a controller system 2, and the steerable instrument 11. The steerable instrument 11 includes an actuation unit 7 and a steerable catheter sheath 100. The steerable catheter sheath 100 includes a multi-segment distal section 3 and a single-segment proximal section 4. The proximal section 4 is connected to the actuation unit 7 via a connector assembly 5. The actuation unit 7 is configured to be detachably mounted to a robotic platform (support platform) 9, as shown in detail on the inset A of FIG. 1A.

The steerable instrument 11 can be configured for a number of medical applications and/or industrial applications. Under medical applications, the steerable instrument 11 can be configured as a robotic endoscope, as a steerable catheter, as a surgical introducer sheath or sleeve that uses principles of kinematic (robotic) navigation for guiding a medical tool through tortuous bodily lumens. Robotic endoscopes can be used for a variety of different diagnostic and interventional procedures including, but not limited to, colonoscopy, bronchoscopy, laparoscopy, video endoscopy, etc. In the case of a video endoscope, the steerable instrument 11 would be configured with a miniature video camera, such as a CCD or CMOS camera, positioned at the distal portion of the bendable body 3, as well as electronic cabling and illumination optics (an optical fiber) extending along the tool channel.

FIG. 1B illustrates an example embodiment of the medical system 1000 in functional block diagram. The catheter sheath 100 has a proximal non-steerable section 4, and a distal steerable section 3 made of the multiple bending segments (e.g., bending segments 14, 13, 12) which are arranged lengthwise along a longitudinal axis (Ax). At least one central lumen or tool channel extends along the length of the catheter sheath 100 and through part of the connector assembly 5. In at least some embodiments, the steerable instrument 11 is controlled by a robotic controller system 2 via the actuation unit 7; the actuation unit 7 is a handheld controller (handle) connected to the proximal section 4 of the catheter sheath 100 by or connector assembly 5. The actuation unit 7 can include any force generating device and a mechanical element respectively used to generate and transmit sufficient actuating force for bending at least one bending segment of the steerable section 3. In that regard, actuation unit 7 may include any device capable of generating and transmitting an actuating force including, for example, a mechanical force, hydraulic force, magnetic force, or pneumatic force. The support platform 9 may include, for example, a robotic arm and a linear stage 91 which serves to guide the steerable instrument 11 (control unit 7, connector assembly 5 and catheter sheath 100) in a moving direction (typically linear movement) for insertion and/or retraction of the catheter sheath 100 with respect to the patient 8.

The controller system 2 generally includes electronic components such as a PID controller and/or a digital signal processor (DSP) device along with suitable software, firmware and peripheral hardware, which are generally known per se to persons having ordinary skill in the art. The controller system 2 can be part of, or is connected to, the navigation system 1 (e.g., a computer or system console). The navigation system 1 includes the necessary software (computer-executable code, programs and applications) executable by a central processing unit (CPU) 190, according to a user's interactions with the system 100 via a user interface 194, to control the steerable instrument 11. Operation of CPU 190 may be implemented by one or more processors in a computer loading and executing a program, or may be implemented by a dedicated circuit (FPGA and ASIC). The user interface 194 may include, for example, a display device 192 (LCD, LED or OLED display) which may include a graphical user interface (GUI) and/or a pointing device and keyboard (not shown), or a touchscreen.

The navigation system 1, the controller system 2, and the actuation unit 7, are operably connected to each other by a network connection or a cable bundle 199 and a data bus system 195. Among other functions, the navigation system 1 can provide a surgeon or other user with a GUI and other information displayed in the image display device 192, so that the user can interact and remotely operate the steerable instrument 11.

The controller system 2 is configured to control the actuation unit 7 which includes a plurality of actuating motors (or actuators) 70-1, 70-2 . . . , 70-M. The number of actuators or motors 70 will depend on the design of the actuation unit 7, and it can include a single (one) actuator or motor that can actuate all driving wires independently, or it could include a number of actuators or motors equal to a number of driving wires 115 so that each actuator or motor can actuate each driving wire individually.

The controller system 2 may also include or be connected to one or more sensors 74. Sensors 74 can include a strain sensor and/or a position sensor which are configured to detect and/or measure compressive or tensile forces (actuating forces) exerted on the driving wires 115 to bend one or more of the segments 12, 13 and 14. Sensors 74 may output a signal 75 corresponding to an amount of compressive or tensile force (an amount of strain) being applied to a driving wire 115 at any given point in time. The signals 75 from the sensors 74 (strain sensor and/or position sensor) for each driving wire are fed into the controller system 2 to control each actuator individually. In this manner, each driving wire can be actively controlled, by a feedback loop, to implement appropriate shaft guidance for navigating the steerable section 3 through intraluminal tortuous paths of a patient's anatomy.

### FIG. 2A-2B: Catheter Sheath Structure

FIG. 2A and FIG. 2B illustrate additional details of the catheter sheath 100, according to an embodiment of the present disclosure. FIG. 2A is a 3D rendering and FIG. 2B is a perspective view of the catheter sheath 100 comprised of a non-steerable proximal section 4 and a steerable distal section 3. The steerable section 3 incudes a plurality of bending segments comprising a proximal bending segment 14, a middle bending segment 13, and a distal bending segment 12. As shown in FIG. 2B, each bending segment is formed of two or more rings (a plurality of rings) cooperatively arranged in a lengthwise direction to form a tubular structure. As shown in FIG. 2A, the tubular structure also includes an outer jacket 80 and a central lumen extrusion 200. The central lumen extrusion 200 includes an inner liner 210 reinforced by a reinforcing structure 220. The inner liner 210 has an inner surface which defines a central lumen or tool channel 150, and an outer surface onto which a plurality of rings are arranged. The rings include a plurality of wire conduits (secondary lumens) through which driving wires 115 and/or support wires 116 are passed. The driving wires 115 are moved by an actuating force to bend one or more segments of the steerable section; the support wires 116 are not actuated.

FIG. 2B illustrates an example of the catheter sheath 100 without the central lumen extrusion 200 and without the outer jacket 80. As shown in FIG. 2B, the plurality of driving wires 115 pass through the proximal section 4, advance through wire conduits of wire-guiding rings 140 of the proximal bending segment 14, pass through wire conduits of wire-guiding rings 130 of the middle bending segment 13, and pass through wire conduits of wire-guiding ring 120 of the distal bending segment 12. Each bending segment of the steerable section is actuated by a set of antagonistic driving wires 115 which operate by a pulling or pushing force (an actuating force) to bend each bending segment independently from each other. Forces F1 and F2 of different magnitude can be applied in the lengthwise direction to separate driving wires to bend the various bending segments in desired directions. A combination of forces F1 and F2 can also be applied to bend a given bending segment in additional directions. To that end, a first set of driving wires 115 may be anchored at an anchor ring 120A at the distal end of the distal segment 12, a second set of driving wires 115 may be anchored at the anchor ring 130A of the middle bending segment 13, and at a third set of driving wires 115 may be anchored at the anchor ring 140A of the proximal bending segment 14.

According to one embodiment, three driving wires 115 may be used to actuate each bending section. In that case, the distal ends of the driving wires 115 in the first set of driving wires can be anchored to anchor ring 120A, the second set of driving wires can be anchored to the anchor ring 130A, and the third set of driving wires can be anchored to the anchor ring 140A. In such example, nine driving wires 115 will pass through the proximal section 4 of the steerable sheath. At each anchor member, it may be advantageous to arrange (to anchor) the driving wires 115 equidistantly around the circumference of each anchor member at strategic locations so as to actuate each bending segment independently in a desired direction. For example, each driving wire 115 can be anchored at equal intervals on the anchor member, e.g., when each bending segment is actuated by three wires, the driving wires would be anchored at 120-degree intervals to be able to actuate each bending segment in substantially any direction (any angle with respect to lumen axis Ax).

As shown in FIG. 2A and FIG. 2B, in the catheter sheath, each bending segment 12, 13, and 14 includes a plurality of ring-shaped wire-guiding members (guide rings), while the proximal non-steerable section 4 is a single piece elongated tubular component. Here, the tubular shaped proximal section 4 and the central lumen extrusion 200 can be made of similar biocompatible polymer materials, such as polyether block amide copolymer (e.g., Pebax® brand produced by Arkema), which is a well known polymer used in the fabrication of catheter shafts. Other medical-grade thermoplastic polyurethane (TPU) and thermoplastic elastomer (TPE) materials can also be used as tubing extrusion materials for medical catheter and endoscope devices that demand precision and consistency. Furthermore, other commonly known catheter tubing materials may be used, including PVC, HDPE, Polyurethane, Nylon, FEP, PFA, ETFE, PTFE (liners), PEEK, TPE, Grilamid® lubricious films, and many others.

### FIG. 2C-FIG. 2D: Ring Structure

Each wire-guiding member (each guide ring) has a plurality of wire conduits (or thru-holes) along the wall of the guide ring. The thru-holes serve as conduits through which wires are guided along the wall of the tubular shaft. Again, the wire conduits could also be formed on the outer surface of each guide ring. The number of wire conduits in each wire-guiding member depends on the bending section in which the wire-guiding member is arranged. The distal bending segment 12 includes a plurality of wire-guiding rings 120; the middle bending segment 13 includes a plurality of wire-guiding rings 130; and the proximal bending segment 14 includes a plurality of wire-guiding rings 140. The distal bending segment 12 is joined to the middle bending segment 13 by an anchor ring 130A; and the middle bending segment 13 is connected to the proximal bending segment 14 by an anchor ring 140A. The proximal section 4 is a non-steerable section, but it does include a plurality of wire conduits extending through the wall (or on the outer surface of the wall). Here, it should be noted that wire conduits are not limited to thru-holes or conduits within the wall itself. In some embodiments, the wire conduits can be formed on the outer surface or the inner surface of the individual rings. Moreover, at least some rings can be formed without thru-holes or conduits.

FIG. 2C shows exemplary representations of annular-shaped guide rings having a central opening or tool channel 150 and secondary lumens or wire conduits (151, 152, 153, 154, 155, 156, 157, 158, 159, etc.,) formed on the wall of the ring surrounding the tool channel 150. The outer surface and the inner surface of each guide ring are shown as circular for ease of illustration, but the actual implementation is no limited thereto. The outer surface and the inner surface of each guide ring structure can have a substantially symmetric and closed polygonal shape, such as a hexagon, octagon, etc.

FIG. 2C shows one wire-guiding ring 120, one wire-guiding ring 130, and one wire-guiding ring 140. The wire-guiding ring 120 includes three wire guiding conduits (151, 154, 157); the wire-guiding ring 130 includes six wire guiding conduits (152-153, 155-156, 158-159); and the wire-guiding ring 140 includes nine wire guiding conduits (151, 152, 153, 154, 155, 156, 157, 158 and 159). In this embodiment, nine driving wires 115 can be arranged through the tubular wall in the proximal section 4. Then, the driving wires continue through the wire conduits of the proximal bending segment 14, and are anchored to an anchor member for each bending segment. The anchoring rings 120A, 130A and 140A are of substantially similar structure as the corresponding wire-guiding rings 120, 130, and 140, respectively. All wire-guiding members and anchoring members include a central opening or tool channel 150, and have a predetermined number of thru-holes (wire-guiding conduits or secondary lumens) arranged around the tool channel 150 substantially parallel to, and equidistant from, the instrument axis Ax.

The number of thru-holes in each ring or wire-guiding member depends on the bending segment to which each ring belongs to. However, in at least some embodiments, some rings may not have thru-holes at all. For example, FIG. 2D illustrates a first ring 120 and a second ring 130 having different structure or function. The first ring 120 has a tool channel 150, but does not have any thru-holes. Instead, the first ring 120 includes a slanted slot 121 on the outer surface the ring. The second ring 130 includes a tool channel 150, a plurality of through holes 151 (secondary lumens) surrounding the tool channel 150, and it also includes an open slot 131 on the outer surface of the ring. The slanted slot 121 in the first ring 120, or the open slot 131 on the second ring 130 can be used to arrange therein either an electronic component (e.g., an EM sensor) or a radiopaque material (radiopaque marker). The EM sensor or radiopaque marker can be used as a reference during image-guided procedures, for example. The axis Ax of each wire-guiding ring (120, 130, 140) or guide ring is arranged substantially coaxial with the sheath axis Ax. Although in geometry the term “coaxial” technically means that two or more three-dimensional linear forms share a common axis, in the steerable sheath illustrated in FIG. 2B and in other drawing disclosed herein, “coaxial” means that two or more components (e.g., the inner liner, the rings, and the outer jacket) share approximately the same axis. In some instances, some components could be paraxial (i.e., having axes parallel to each other) rather than exactly coaxial. However, for paraxial components where the distances between the axes are small, it can be considered that the axes of such components are effectively coaxial.

In referring to FIG. 2A-FIG. 2D, it should be understood that not all thru-holes are used for driving wires no. At least some of those thru-holes are used to pass an electrical cable, some through holes are empty, and some have support wires that are not drive wires. That is, according to at least one embodiment, the thru-holes of each guide ring can have several uses, for example, some thru-holes may contain a control wire (driving wire), some may contain a support wire which transmits no force, some are left empty, some may pass an optical fiber, some may have an electrical cable, and some may have an electronic component such as a load cell or sensor. The rings for the steerable section 3 can be made of biocompatible thermoplastic polymer similar to that used for the central lumen extrusion or the proximal section 4.

In at least some embodiments, the rings 120, 130, 140 are made of transparent or translucent material that can facilitate bonding to the central lumen extrusion 200. For example, the rings 120, 130 and 140 are made of Pebax at a natural clear color. With a transparent material, the rings can be bonded on the central lumen extrusion with the bonding process associated with light energy transfer. Therefore, even miniature rings can be bonded with consistent boding quality as part of the manufacturing process. For example, UV adhesive can be used to bond the rings on the central lumen extrusion. In another design example, the rings 120, 130 and 140 are made of Pebax at the natural clear color while the central lumen extrusion includes carbon black on the outer layer, and the black color is advantageous to absorb laser light more efficiently. This particular combination of the rings and the central lumen extrusion allow bonding the rings on the central lumen extrusion with laser welding without affecting the inner surface of the central lumen. Laser welding provides consistent strong bonding between the rings and the central lumen extrusion. To minimize unnecessary heating to the other parts from the bonding area, it is preferable that the central lumen extrusion includes carbon black only in the outer layer of the central lumen extrusion. Other examples of biocompatible medical-grade translucent materials are described in pre-grant patent application publication US 20160220735 which is incorporated by reference herein for all purposes.

Referring back to FIG. 1A and FIG. 1B, the handle or connector assembly 5 provides an electromechanical interface between the proximal section 4 and the actuators in actuation unit 7. For example, the connector assembly 5 may provide mechanical, electrical, and/or optical connections, and other data/digital connections for interfacing the steerable instrument ii with the controller system 2 and the navigation system 1. The handle or connector assembly 5 may also provide an access port 55 which can be used by a surgeon or other operator to insert instruments or end effectors through the tool channel 150. For example, the access port 55 can be used to insert small instruments, such as small forceps, needles, or electrocautery instruments and the like. In addition, the connector assembly 5 may include one or more dials or control wheels 52 for manual control (bending or steering) of at least one segment of the steerable section. In some embodiments, the bendable body 3 may include more that one tool channel 150, where at least one of those channels can be used for passing liquid and/or gaseous fluids, and another channel can be used for passing tools or imaging devices.

In operation, the navigation system 1 and the controller system 2 are communicatively-coupled via the data bus 199 to transmit and receive data to and from each other. The navigation system 1 is also connected to, and communicates with, external equipment such as a computed tomography (CT) scanner, a fluoroscope imager, an image server (not shown in FIG. 1A), etc., which are external of the medical system woo. The image server may include, but is not limited to, a DICOM™ server connected to a PACS (Picture Archiving and Communication System) or medical imaging system which may include, but is not limited to, one or more of the CT scanner, a magnetic resonance imaging (MRI) scanner, or a fluoroscope, etc. The navigation system 1 processes data provided by the controller system 2, data provided by images stored on the image server, or data provided by images from the CT scanner or the fluoroscope. The navigation system 1 displays images and other medical information in an image display device 192 to aid the user 10 in performing a medical procedure.

For a medical procedure where the steerable instrument ii will be used, medical images (e.g., from the CT scanner) are pre-operatively provided to the navigation system 1. With the navigation system 1, a clinical user creates an anatomical computer model from the images. In a particular example embodiment of FIG. 1A, the anatomy can be the lung airways of patient 8. From chest images received from the CT scanner or PACS system, the clinical user can segment the lung airways for clinical treatments, such as a biopsy. After the navigation system 1 generates a map of the lung airways, the user can also use the navigation software system to create a plan to access a lesion for the biopsy. The plan includes the target lesion and a trajectory (navigation path) through the airways to insert the bendable body 3 (steerable sheath) of the steerable instrument 11.

The controller system 2 includes firmware, control circuitry and peripheral hardware to control the steerable instrument 11, the insertion unit 9, and a field generator 6 (e.g., an electromagnetic (EM) field generator). The controller system 2 is communicatively coupled with the actuation unit 7, the insertion unit 9, the EM field generator 6, and a man-machine interface (e.g., a gamepad controller not shown in FIG. 1A-FIG. 1B). In this manner, the controller system 2, in coordination with the navigation system 1, controls the overall functions of the steerable instrument 11 and the insertion unit 9.

The steerable instrument 11 includes the bendable body 3, the handle or connector assembly 5, and the actuation unit 7. The actuation unit 7 is configured to bend one or more the proximal bending segment 14, the middle bending segment 13, and the distal segment 12 via the connector assembly 5 according to commands from the controller system 2, and based on the navigation plan provided by navigation system 1.

According to one embodiment, either during insertion or retraction of the steerable instrument 11, the controller system 2 may control the linear stage 91 of insertion unit 9 to move the bendable body 3 along the center line of a lumen (e.g., an airway) in a desired trajectory followed by active control of the bending segments. This is similar to known shaft guidance techniques used to control robotic guided catheters or endoscopes with the goal of forcing the flexible shaft of the sheath to keep to a desired trajectory. In one example, when using the navigation system 1, the steerable instrument 11 is robotically controlled to advance the sheath through a lumen while sensors 74 measure the actuation force, insertion depth, the angulations of user-controlled steerable segments, etc., to obtain trajectory information. The trajectory information is stored in a memory of the system and continuously updated. After a short advance in insertion or retraction distance, the shape of the bendable body 3 is corrected by adjusting (actuating) one or more of the bending segments in such a way that the new shape closely matches the desired trajectory. This process is repeated until a target area is reached. The same process can be applied when the steerable instrument is controlled to withdraw the bendable body 3 from the patient. This process is similar to the navigation process described in, e.g., US 2007/0135803, which is incorporated by reference herein for all purposes. Additional details for driving a snake-like robot include the control methods for actuation, as described in applicant's previous patent application publications US 2015/0088161, US 2018/0243900, US 2018/0311006, and US 2019/0015978, which are also incorporated by reference herein for all purposes. To improve the navigation process, it is advantageous to reinforce the inner liner of the central lumen or tool channel 150.

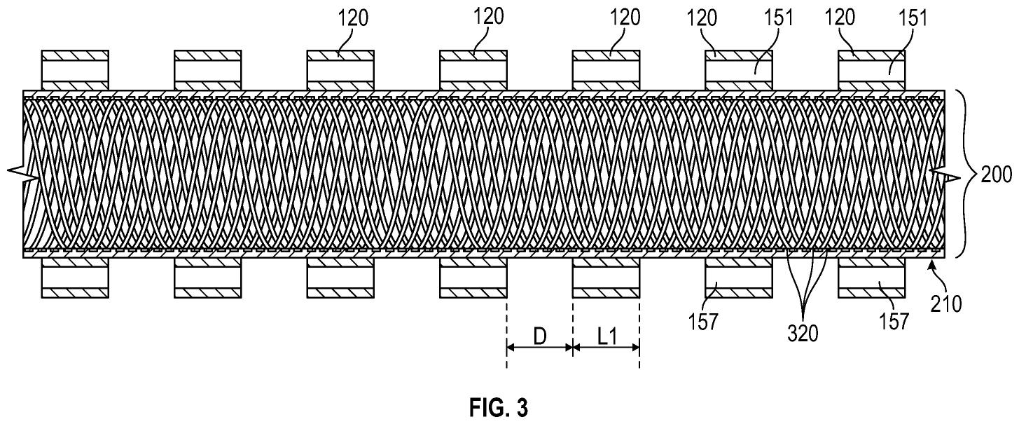

### FIG. 3-5: Central Lumen Extrusion with Reinforcing Structure

According to an example embodiment, the steerable sheath of the bendable body 3 includes a central lumen extrusion with a reinforcing structure. The central lumen extrusion is an inner liner made of one or more layers of polymer material and reinforced to increase its hoop strength, pushability, steerability, and kink resistance, by adding a reinforcing structure made of metal, metal alloys, polymer materials, or combinations thereof. The increased hoop strength will reduce the deflection of the inner liner thereby preventing that an instrument passing through the central lumen could catch on the rings of the sheath. The reinforcing structure can be designed to not impact the overall flexibility of the central lumen extrusion, but to enhance the hoop strength. The material used for the reinforcing structure could be of any biocompatible metal or polymer. The reinforcing structure for the central lumen extrusion includes one or more of a braid structure, a coil structure, or a laser cut tube (hypotube) structure combined with one or more layers of polymer, preferably elastomeric polymer. In this manner, the central lumen structure can include one or more of a braided reinforced inner liner, a coil reinforced inner liner, and a laser cut tube reinforced inner liner.

FIG. 3 illustrates an example embodiment of a central lumen extrusion 200 reinforced by a braid reinforcing structure 320. The central lumen extrusion 200 in FIG. 3 shows an inner liner 210 with the braid reinforcing structure 320 and a plurality of guide rings 120 collectively arranged (bonded) to the outer layer of the inner liner 210 to form one or more bending segments. Each guide ring 120 includes a plurality of wire-guiding conduits (wire conduits or secondary lumens). In FIG. 3, each guide ring 120 is illustrated with a first wire guiding conduit 151, and a second wire guiding conduit 157 (refer to FIG. 2C). Two consecutive guide rings 120 are arranged at a distance D1 from each other to form a gap therebetween. The gap distance D1 is substantially equal to or smaller than a length L1 of each guide ring 120. Here, the length L1 and gap distance D are measured in the lengthwise direction (i.e., parallel to the longitudinal axis Ax).

According to one or more embodiments, a ratio of the guide ring length L1 to the gap distance D can be tailored to achieve desired sheath characteristics including hoop strength and bending flexibility. For example, the length L1 of each guide ring and the gap distance D between the each pair of consecutive guide rings is in a range from 0.5 mm to 1.5 mm, or in a range from 0.75 mm to 1 mm. A ratio of the length of each of the guide rings to the gap distance (Li/D) between the consecutive guide rings is in a range of 3 to 0.3, or in a range of 2 to 0.5, or in a range of 1.5 to 1. Furthermore, the length L1 of each guide ring and the gap distance D between the each pair of consecutive guide rings is respectively 1 mm and 0.5 mm, or 0.75 mm and 0.75 mm, or 0.75 mm and 0.5 mm. These dimensions (unless otherwise noted) apply to all embodiments.

In FIG. 3, the reinforcing structure 320 is made of braided strands (filaments or filars) of metal and/or hard polymer material. As used herein the term “braid” or “braided” refers to a structure or pattern formed by interlacing or weaving two or more strands or filars of flexible material such as flexible wire. The strands of wire can have a circular (round) or rectangular (flat) cross-section. The inner liner 210 is reinforced by the braided structure 320 so as to have sufficient thickness and hoop strength to easily bend, while maintaining torsional resistance and preventing an instrument from catching on the guide rings 120.

For a braid reinforced central lumen extrusion, a variety of materials can be used to enhance the properties of the tubular shaft depending on the performance characteristics that are to be achieved (e.g., torsional and kink resistance, increased flexibility, enhanced hoop strength, etc.). According to at least one embodiment of the present disclosure, the reinforced central lumen extrusion of inner liner 200 has three components, an inner layer, the braid structure, and an outer layer. These components are combined to obtain specific characteristics in terms of hoop strength, flexibility, and kink and torsional resistance. At the same time, the three components must be combined to satisfy desired dimensions (e.g., wall thickness of the reinforced liner) and manufacturing/assembly tolerances. In that regard, it must be noted that the overall outer diameter (OD) and the size of the inner diameter (ID) of the steerable sheath may necessarily limit the number of polymer layers and the type/thickness of the reinforcing structure that can be used. For example, since the braided filaments (braid wire) must cross over each other repeatedly to form the braid structure, the overall thickness of the inner liner 200 will be determined by adding the thickness of the inner layer plus at least twice the diameter of the braided wire and plus the thickness of the outer layer. Therefore, a larger diameter braid wire could provide more stiffness and torsional resistance, but it may increase the minimum wall thickness, and this will impact flexibility. On the other hand, a flat wire braided to form the reinforcing structure could maintain a minimal wall thickness and provide certain increase of flexibility, but may not provide significant torsional resistance.

Another aspect to consider for the braid reinforcing structure 320 is the pick count. The pick count is expressed in picks per inch of length (PPI), and it represents the number of times the braid wire crosses for every inch of the shaft length. According to one example embodiment, the braid wire used in current prototypes is 304 stainless steel (304SS) flat wire of 0.0005×0.003 inches cross-section. The braid pattern used is 130 PPI; meaning that for every inch of the braid, there are 130 repeating cells (picks). The higher the pick count, the more flexible the braid will be in bending. Typical braid patterns have a 70-80 PPI. In the present disclosure, since the central lumen extrusion must be bendable with relatively low force inputs, a much higher pick count has been prototyped and yielded good results. The braid density will depend on the size of the flat wires as well as the size (inner diameter and outer diameter) of the central lumen extrusion. For a catheter sheath having a central lumen extrusion with ID of size 6 to 10 French, the braid density would accordingly range between about 50 PPI and about 200 PPI. Additionally, to provide greater flexibility near the distal end of the central lumen extrusion and greater axial rigidity towards the proximate end, the pic rate may be varied along the length of the central lumen. For example, depending on the application, the braid reinforcing structure 320 may have 50-200 pics per inch, and this value may vary along the length of the inner liner 210 so as to create increasingly flexible sections in a direction from the proximal to the distal end.

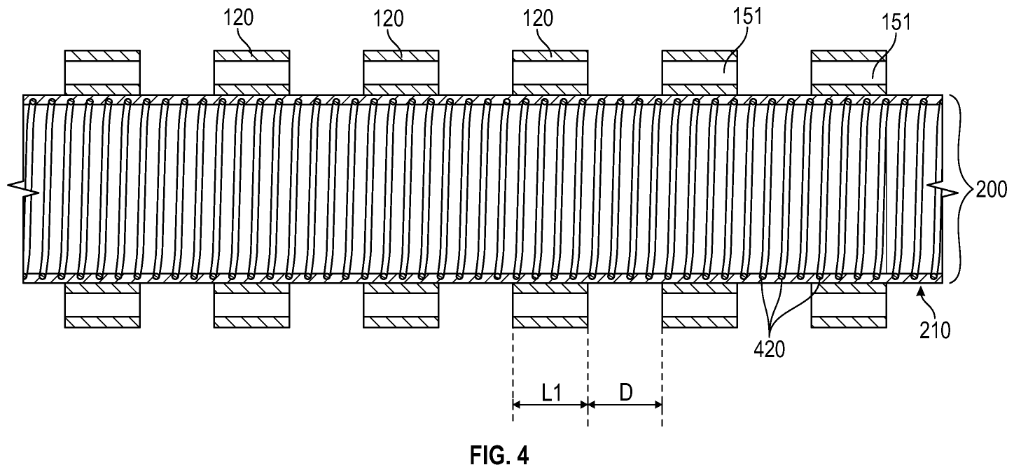

FIG. 4 illustrates an example embodiment of a central lumen extrusion 200 reinforced by a coil reinforcing structure 420. The central lumen extrusion 200 in FIG. 4 shows an inner liner 210 with the coil reinforcing structure 420 and a plurality of guide rings 120 arranged collectively to form one or more bending segments. The coil reinforcing structure 420 is made of a coiled wire of metal material and/or coiled rods of polymeric material. The rods of polymeric material can include single strands of polymeric fiber and/or spool twisted multi-fiber rods that are coiled in specific patters (e.g., coiled at varying pitch) to obtain the desired characteristics of hoop strength and bending flexibility. The coil reinforcing structure 420 can be manufactured with a variety of wire, filaments, filars or strands having flat, rectangular, square and/or round cross-section, and can be made of metal or polymer-based materials, such as stainless steel, nitinol, fiberglass, carbon fiber, nylon, fluorocarbon, PEEK, PET, PEN, Kevlar®, and others. The arrangement of the guide rings 120 is the same as described with respect to FIG. 3 and remaining embodiments. According to one example embodiment, the wire to be used in the coil reinforcing structure 420 is a 0.001″×0.003″ 304SS wire (substantially flat wire), but circular or round wire of similar dimension may also be used. A coil reinforcing structure 420 can provide the necessary hoop strength and be more flexible than a braid reinforcing structure, but a braid reinforcing structure provides better torsional stiffness. Therefore, in some embodiments, the catheter sheath 100 can have more than one type of reinforcing structure.

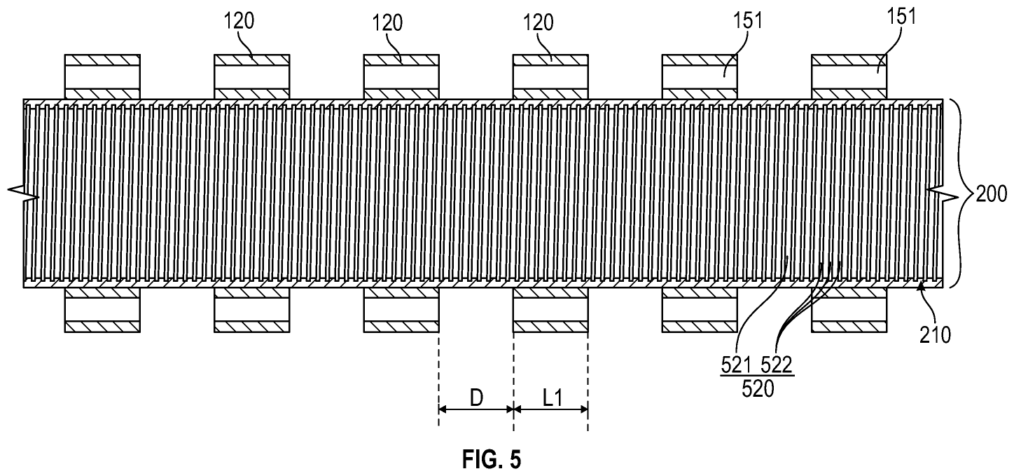

FIG. 5 illustrates an example embodiment of a central lumen extrusion 200 reinforced by a laser-cut tube structure 520. The central lumen extrusion 200 of FIG. 5 shows an inner liner 210 with the laser-cut reinforcing structure 520, and a plurality of guide rings 120 arranged collectively to form one or more bending segments. The laser-cut reinforcing structure 520 is made of a metallic or polymeric tube 521 which is laser cut to form slots 522. The arrangement of the guide rings 120 is the same as described above with respect to FIG. 3 and FIG. 4. According to one example embodiment, the tube 521 can be made of 304SS or nitinol, or it can be made from a polymer-like polyimide. In an embodiment, the tube 521 can be a conventional hypotube laser-cut with specific slot patterns to provide hoop strength and lateral flexibility. A continuous and/or interrupted spiral cut pattern of slot cuts 522 can be developed to provide the desired combination of hoop strength, flexibility in bending, and resistance to twisting of a catheter. With a laser-cut reinforcing structure 520 having an interrupted laser-cut pattern, the laser-cut tube can make the central lumen extrusion more resistant to compression and elongation than a coil or a braid reinforcing structure can. Therefore, the catheter sheath 100 of a steerable continuum robot can have at least some sections of the catheter sheath made of a central lumen extrusion 200 having multiple layers reinforced by an embedded laser-cut tube as illustrated in FIG. 5.

Naturally, the catheter sheath 100 can have a combination of reinforcing structures alternately arranged along the length of the central lumen extrusion 200; the reinforcing structures being selected from the laser-cut tube, the coil, and/or the braid reinforcing structure described in the foregoing embodiments. In one embodiment, each bending segment can have a different reinforcing structure. For example, the proximal segment 140 can have the central lumen extrusion 200 reinforced by the laser-cut tube reinforcing structure 520, the middle segment 130 can have the central lumen extrusion 200 reinforced by the coil reinforcing structure 320, and the distal segment 120 can have the central lumen extrusion 200 reinforced by the braid reinforcing structure 420. The reinforcing structure can be interchangeably adapted to each being segment according to the desired application and need for hoop strength and flexibility.

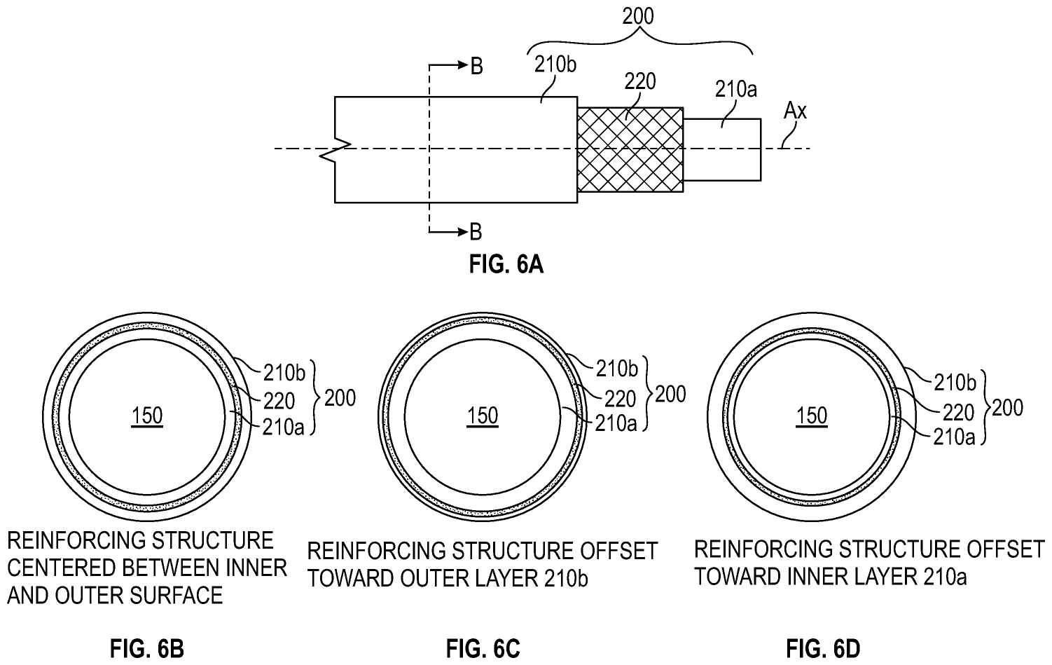

### FIG. 6A-6D: Offset Central Lumen Reinforcing Structure

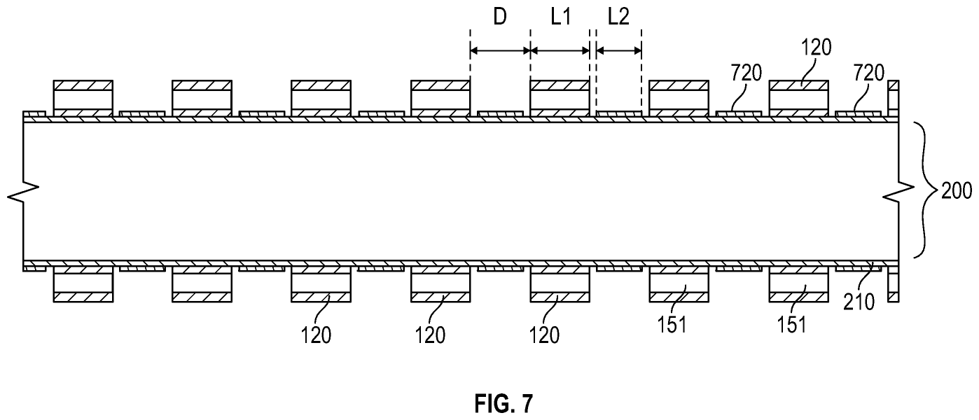

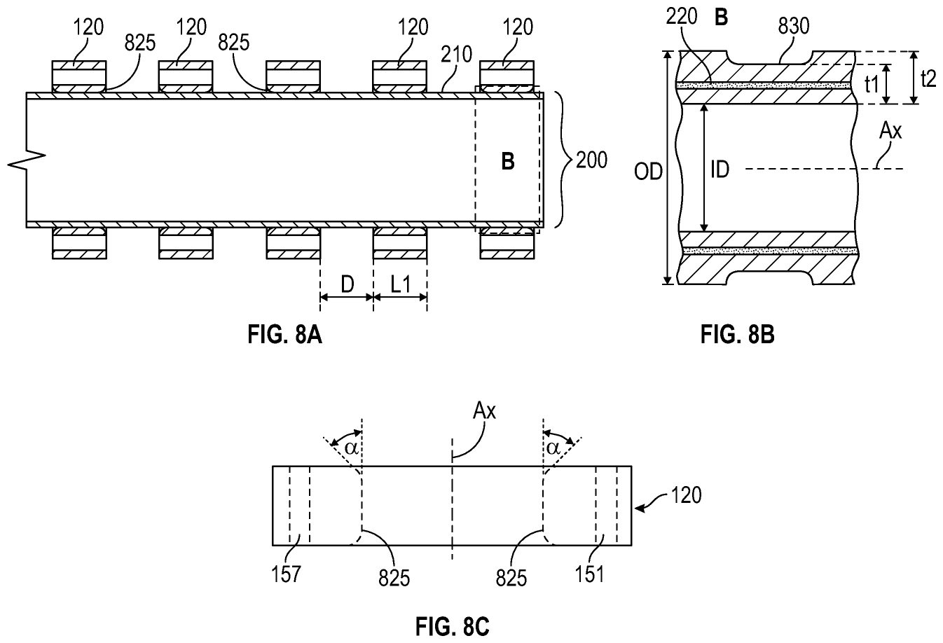

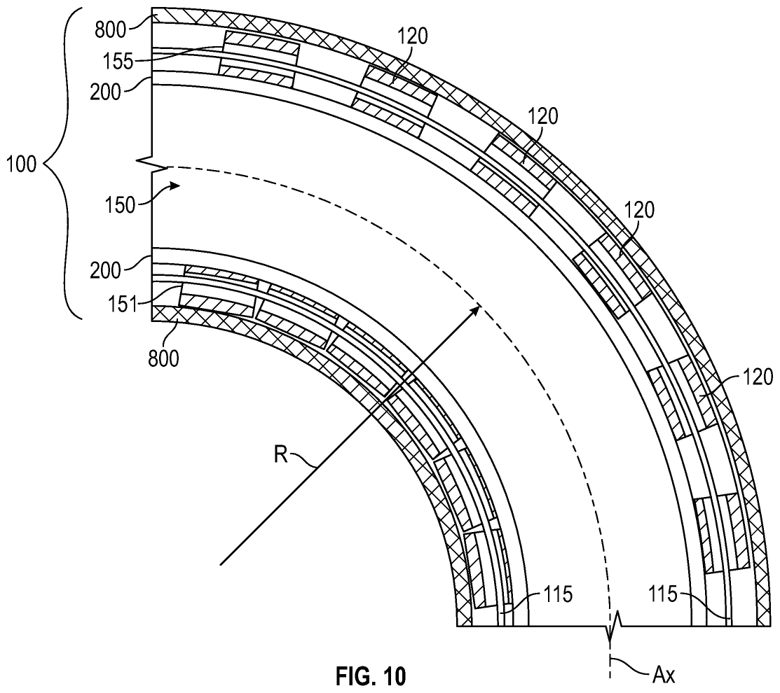

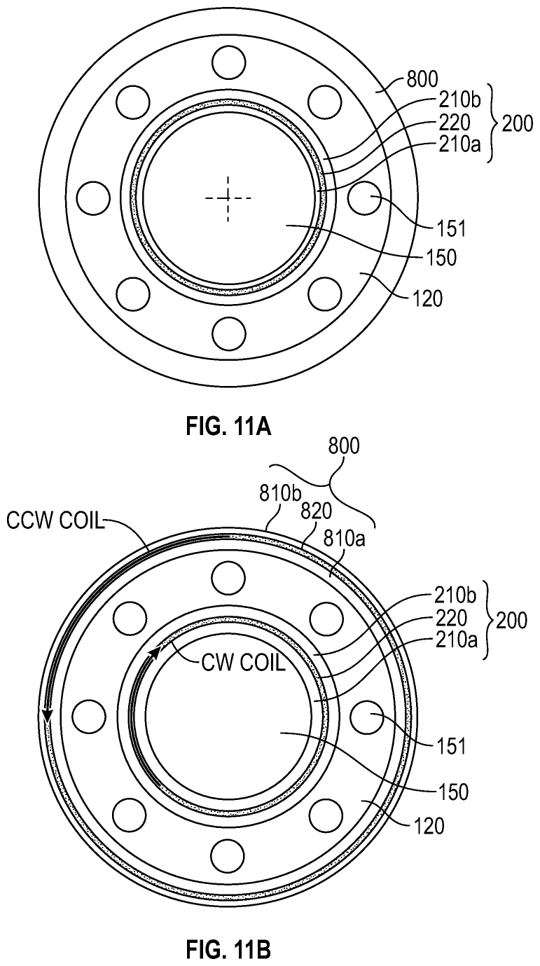

FIG. 6A shows an example embodiment of the central lumen extrusion 200. The central lumen extrusion 200 includes an inner layer 210a, a reinforcing structure 220, and an outer layer 210b, which are arranged in this order substantially concentric to, and an equidistant from, the lumen axis Ax. The inner layer 210a and the outer layer 210b make up the inner liner 210 shown in FIG. 3, FIG. 4, FIG. 5, FIG. 7, FIG. 8, FIG. 10, and FIG. 11A-11B.

The reinforced central lumen extrusion 200 can be made plastic by any known process including, but not limited to, by injection molding, blow molding, or extrusion. For example, the processes described in the above referenced U.S. Pat. Nos. 7,550,053 and 7,553,387, and publication US 2009/0126862 can be used for making the reinforced central lumen extrusion of any of the embodiments disclosed herein. While a reinforced central lumen extrusion could be made from a continuous process to form the entire central lumen extrusion in a single process that combines the reinforcing structure 220 and the inner/outer polymer layers (210a, 210b), the central lumen extrusion can also be made in discrete steps using two or more different extruded parts (e.g., a first part being the outer layer 210b extruded in a first step, and a second part being the inner layer 210a extruded in a second step). Thereafter, a third step will be preformed that will sandwich the reinforcing structure 220 in between the inner layer 210a and the outer layer 210b.

The process of making the inner layer in discrete steps can provide a configuration that allows for combinations of durometers that could benefit the resulting central lumen extrusion. For example, the first part being the outer layer 210b can have a different material and different durometer than the second part being the inner layer 210a. In addition, the length of the inner layer 210a and/or the length of outer layer 210b can be made in separate parts having different materials/durometers.

FIG. 6B shows a cross-sectional view of the reinforced central lumen extrusion 200, as seen along section B-B of FIG. 6A. As shown in FIG. 6B, the reinforcing structure 220 can be arranged between the inner layer 210a and the outer layer 210b halfway (in the middle of) the thickness of the wall. However, to obtain the specific requirement of enhanced hoop strength and flexibility, the durometer of the materials used for the inner layer 210a and for the outer layer 210b can be alternated, adjusted, modified, mixed, doped, and the like. For example, according to one embodiment a higher durometer material could be used for the inner layer 210a than for the outer layer 210b, or vice versa. In other words, the central lumen extrusion incudes two layers (an inner layer and an outer layer) and a reinforcing structure arranged between the two layers. The two layers are jointed by any known process to secure the reinforcing structure (braid or coil or laser-cut tube layer) therebetween. The material of the inner layer is or includes low friction material, and an example of the inner layer material is ePTFE. The outer layer material can be low or high durometer material; one example of the outer layer material is Pebax®. An advantageous effect is that this combination of features can increase torsional stiffness of the catheter body as a whole, while maintain good flexibility and hoop strength of inner tube for tight curvatures of bending.

According to one example embodiment, the central lumen extrusion 200 includes an inner layer 210a made of high durometer elastomer, an outer layer 210b made of low durometer elastomer, and braid or coil or laser-cut tube reinforcing structure 220 arranged between the inner and outer layers. The higher durometer inner layer 210a can be tailored to increase lubricity and/or can be made to increase resistance to damage from the tools and instruments passing through the lumen. Advantageously, since the inner layer 210a of the central lumen extrusion is closer to the central axis Ax of the lumen or tool channel 150, the higher durometer inner layer 210a will experience less bending strain. In this case, the reinforcing structure can be offset towards the inner surface. Therefore, the resulting reinforced center lumen extrusion meets the desired enhanced loop strength and flexibility.

According to another embodiment, the central lumen extrusion 200 includes an inner layer 210a made of low durometer elastomer, an outer layer 210b made of high durometer elastomer, and a braid or coil or laser-cut tube reinforcing structure 220 arranged between the inner and outer layers. Advantageously, since the outer layer 210b is bonded to the guide rings 120, when the sheath is bent the higher durometer outer layer 210b provides enhanced bending flexibility and sufficient hoop strength. Examples of durometer values include high durometer in a range of about 63 D to 72 D Shore, and lower durometer in a range of about 25 D to 35 D Shore.

Low durometer polymers tend to have a tacky surface. Therefore, when low durometer material is used in the inner layer 210a certain measures should be taken. Specifically, because of the tacky surface, greater frictional force may be encountered during passage of instruments through the central lumen or tool channel 150. If this frictional force is reduced, the central lumen extrusion 200 will deflect less, and the instrument has a greater facility to pass without catching on the guide rings. Therefore, according to an example embodiment of the present disclosure, friction of the inner layer is reduced by selecting a different material that is similar in stiffness but provides increased lubricity. To that end, in an alternate embodiment, a lubricious additive could be added only to the inner layer 210a of the central lumen extrusion to increase lubricity for the passage of instruments. When a lubricious additive is added only to the inner layer (or more specifically to the inner surface), bonding the outer layer of the reinforced lumen extrusion to the guide rings would not be affected. Therefore, according to one embodiment, the inner layer 210a of the central lumen extrusion 200 could be made of, or coated with, a more lubricious material than the outer layer 210b. A more lubricious material can include expanded PTFE (ePTFE) or other similar fluoropolymer liner or coating.

A further alternate embodiment of the present disclosure uses a lubricious additive compounded into the resin or material of the inner layer 210a of the central lumen extrusion 200. In this embodiment and other embodiments, the reinforcing structure 220 can be one or more a braided reinforcing structure 320, a coiled reinforcing structure 420, and a laser-cut tube reinforcing structure 520. For example, the steerable section of the catheter sheath can have the first bending segment reinforced by a braid structure, the second bending segment reinforced by the coil structure, and the third bending segment reinforced by the laser-cut tube structure. Regardless of the reinforcing structure used, the lubricious additive compounded into the resin or material of the inner layer 210a enhances lubricity of the reinforced central lumen extrusion. In addition, at the time of a procedure, a lubricant can be placed on the instrument or flushed through the central lumen extrusion to decrease frictional forces. According to at least some embodiments, the inner and outer layers can be extruded from the same resin, like Pebax®, but the inner layer can have an additive compounded into the base resin to increase lubricity. There are various additives commercially available, including for example, Propell® (Foster Corp), Mobilize (Compounding Solutions), and Pebaslix® (Duke Empirical Inc.), among many others.

In this manner, the inner layer 210a provides the central lumen extrusion 200 with a high degree of lubricity on the inner diameter that facilitates the passage of diagnostic or therapeutic devices through the central lumen without being caught by the guide rings of the skeleton structure. The inner layer 210a with a more lubricious material provides a smooth, yet rigid inner surface to promote smooth tool handling.

To further enhance hoop strength and bending flexibility, the reinforcing structure 220 is offset with respect to the central lumen extrusion layers. FIG. 6C and FIG. 6D respectively show a cross-sectional view of the reinforced central lumen extrusion 200, where the reinforcing structure 220 is offset with respect to the inner and outer layers. Conventionally, a reinforced extrusion shaft has its reinforcement structure in the center of the wall (i.e., in the center between the inner layer and the outer layer), and the durometer of the shaft may vary in the lengthwise direction of the sheath. In contrast, according to at least one embodiment of the present disclosure, the reinforcing structure 220 is offset either toward the outer layer 210b, as shown in FIG. 6C, or toward the inner layer 210a, as shown in FIG. 6D.

The offset of the reinforcing structure can be chosen according to the desired characteristics for the resulting reinforced central lumen extrusion. For example, according to the embodiment of FIG. 6C, the reinforcement structure 220 could be offset towards the outer layer 210b (i.e., the reinforcing structure 220 is closer to the OD than to the ID) to reduce risk to exposing the braid or coil or tube to the tool channel 150. This means that a thickness of the inner layer is larger than a thickness of the outer layer. This can result in improvement of the lifetime for multiple tool usage. On the other hand, according to the embodiment of FIG. 6D, the reinforcement structure 220 could be offset towards the inner layer 210a (i.e., the reinforcing structure 220 is closer to the ID than to the OD) to provide more material for thermal bonding of the guide rings of the skeleton structure to the outer layer and to prevent disruption to downstream processing. In this case, it means that a thickness of the outer layer is larger than a thickness of the inner layer. This can improve compatibility with downstream processes, which is bonding the guide rings to the inner tube with either reflow/laser welding process. In addition, providing the reinforcing structure closer to the ID will increase hoop strength of the central lumen.

Further more, as discussed in more detail further below, both the central lumen extrusion (inner liner) and the outer jacket extrusion can be reinforced with a reinforcing structure.

In any of the foregoing embodiments, whether the reinforcing structure is offset or not, the central lumen extrusion can be further enhanced when only the outer layer includes a radiation absorbing additive material, such as carbon black. Carbon black is a form of paracrystalline carbon used in extrusion processes as reinforcing filler in rubber products, especially tires. Carbon black is also a radiation-absorbing material, which absorbs strongly from the ultraviolet to the infrared (about 350 nm to about 1100 nm) wavelengths of light. Therefore, in the present disclosure, the outer layer of the central lumen extrusion is formed with thermoplastic elastomer mixed with carbon black to improve compatibility with downstream process, which is the bonding of the guide rings to the inner liner with laser welding or reflow. In one embodiment, the outer layer of the central lumen extrusion may be formed by extrusion of a polyurethane elastomer comprising from about 0.5% to 10% by weight of carbon black, or containing about 2% to 5% of carbon black.

When the outer layer is made of thermoplastic elastomer (TPE) mixed with carbon black, the central lumen extrusion in any of the above embodiments can become an obscured (black) colored layer only on the outer layer, and this can allow safer use of laser welding to bond the guide rings to inner liner. This advantageous effect can be attained because TPE mixed with carbon black absorbs laser energy more than TPE alone. In this manner, since only the outer layer is obscured (black), the heat from laser welding will bond the guide rings to the reinforced central lumen extrusion while preventing the heat from penetrating to inner layer. The resulting effect is that laser welding will provide an effective and secure bonding of the guide rings to the central lumen extrusion outer surface and will keep the inner surface of the lumen smooth for tool channel.

The use of carbon black additive material in the outer layer of the central lumen extrusion is for coloring (obscuration) purposes, but it is considered important to achieving appropriate hoop strength. This is because in the assembly process, only the outer layer becomes black color and absorbs laser energy more efficiently, so that the outer layer is heated selectively at the locations where the guide rings are bonded by welding to the central lumen extrusion. Since the inner layer is not heated, it is possible to reduce the risk of overheating or melting the inner layer and to prevent affecting the smoothness of the central lumen.

### FIG. 7: Central Lumen Extrusion with Reinforced Outer Diameter

According to a further example embodiment, the central lumen extrusion can be reinforced by adding a reinforcing structure to the outer diameter (OD) or outer surface thereof. FIG. 7 illustrates an example embodiment of a reinforced central lumen extrusion 200. According to this embodiment, the central lumen extrusion 200 includes an inner liner 210 made of one or more layers of polymer similar to the embodiments described above. In this embodiment, the inner liner 210 is reinforced by a plurality of thin-walled rings (reinforcing rings) 720 formed in between the already attached guide rings 120. The thin-walled rings 720 can be provided in addition to, or in stead of, embedding the reinforcement structure (braid, coil, or laser-cut tube) within the layers of the central lumen extrusion. The thin-walled rings 720 have a smaller diameter and smaller length and the guide rings. In addition the thin-walled rings 720 can be made of a material having a unique Poisson ratio that specifically would allow the central lumen extrusion to bend without changing its diameter dimensions. The thin-walled rings 720 could be made of hard polymer materials and manufactured according to known processes, for example, as disclosed by U.S. Pat. No. 7,815,975, which is incorporated by reference herein. However, to allow sufficient space for bending of the catheter sheath while providing enhanced hoop strength, a length L2 of the thin-walled rings 720 must be tailored to achieve the desired flexibility and torsional characteristics. In that regard, the length L2 is smaller than the length L1 of the guide rings 120, and smaller than the gap distance D between consecutive guide rings 120. In at least some embodiments, one or more thin-walled rings 720 made of radiopaque material (e.g., platinum, gold, or radiopaque polymers) can be used. This would allow identification of the central lumen and/or identification of the one or more bending segments under an image guided procedure. The thin-walled rings 720 can be ePTFE rings arranged in the “ring gap” to bolster the inner liner strength and prevent possible damage of the sheath by a passing tool when the catheter sheath is bent.

### FIG. 8A-8C: Reinforced Central Lumen Extrusion with Grooved Outer Surface and/or Guide Rings with Chamfered Edges

According to a further alternate embodiment, the central lumen extrusion can be reinforced by increasing the wall thickness of the inner liner and providing a reinforcing structure in the manner in which the guide rings are bonded to the extrusion's outer surface. FIG. 8A illustrates an example embodiment of a reinforced central lumen extrusion 200. FIG. 8B shows a detailed view of an area B of FIG. 8A showing an example of how a guide ring can be attached to the central lumen extrusion. FIG. 8C shows an example embodiment of a guide ring 120 with chamfered or beveled or rounded inner edges 825.

According to this embodiment, the central lumen extrusion 200 has an inner liner 210 made of one or more layers of polymer material similar to the previous embodiments. The central lumen extrusion 200 is reinforced by slightly increasing the thickness or number of layers making up the inner liner 210, and by forming a reinforcing structure in the manner in which the rings 120 are attached to the outer layer of the central lumen extrusion. In one embodiment, the inner liner 210 is reinforced with one or more of a braid, coil, or laser-cut tube as in the previous embodiment, but the rings are modified to have chamfered inner edges 825. In other embodiment, the inner liner 210 is modified to form grooves 830 (grooved sections) where the rings 120 with chamfered edges 825 are arranged.

More specifically, to provide the desired characteristics of enhanced hoop strength and lateral flexibility, a plurality of grooves 830 is formed on the outer surface of the inner liner 210. As shown in the area B of FIG. 8A (shown expanded in FIG. 8B), the groves 830 can be formed at specific locations where the guide rings 120 are bonded or pressure fitted to the central lumen extrusion. As an alterative to forming grooves 830 or making the inner edges 825 beveled, at least the inner edges 825 of guide rings 120 can be made of a softer (lower durometer) material than the material of the outer layer 210b of the inner liner 210.

In this embodiment, in order to have the same enhanced performance but with an increased inner diameter (ID), the inner liner 210 can be designed with grooves 830. Grooves 830 can be made by laser cutting, heat shrinking or reflowing the outer surface of the inner liner 210 at locations where the central extrusion contacts the inner diameter of the guide rings 120. In this manner, the wall thickness t2 of the liner 210 is lowered to a thickness t1 (made thinner) only in the sections where grooves 830 are formed. That is, the wall thickness of the central lumen extrusion 200 is thinner where inner liner 210 contacts the guide rings 120. This is done because the guide ring 120 adds wall strength to the inner liner 210, and where the central lumen extrusion does not contact the guide ring 120, the wall thickness is thicker. The inner diameter (ID) of the central lumen extrusion is continuously uniform and smooth or has no meaningful dimension change along its length. On the other hand, the outside diameter of the inner liner 210 has meaningful change in its dimension along its length. The liner “relief or groove” 830 may also have radiused (rounded) or beveled end surfaces. This will help relieve concentration of strain on a single point where the outer surface of the inner liner 210 meets the plane of the guide ring 120. In addition, the rings 120 can have the inner edges (the edges of the inner surface thereof) rounded or beveled at an angle alpha of about 30 to 45 degrees, as shown in FIG. 8C. Either the beveled inner edges 825 of the guide rings 120 and/or the rounded end surfaces of grooves 830 on the outer surface of the inner liner 210 make the sheath more durable during repeated bending/use while enhancing the hoop strength and lateral flexibility. Advantageously, the inner edges 825 of the guide rings 120 are chamfered or radiused or beveled to make it less likely for the instrument passing through the central lumen to catch on the edge of the guide ring 120 when the catheter sheath is bent.

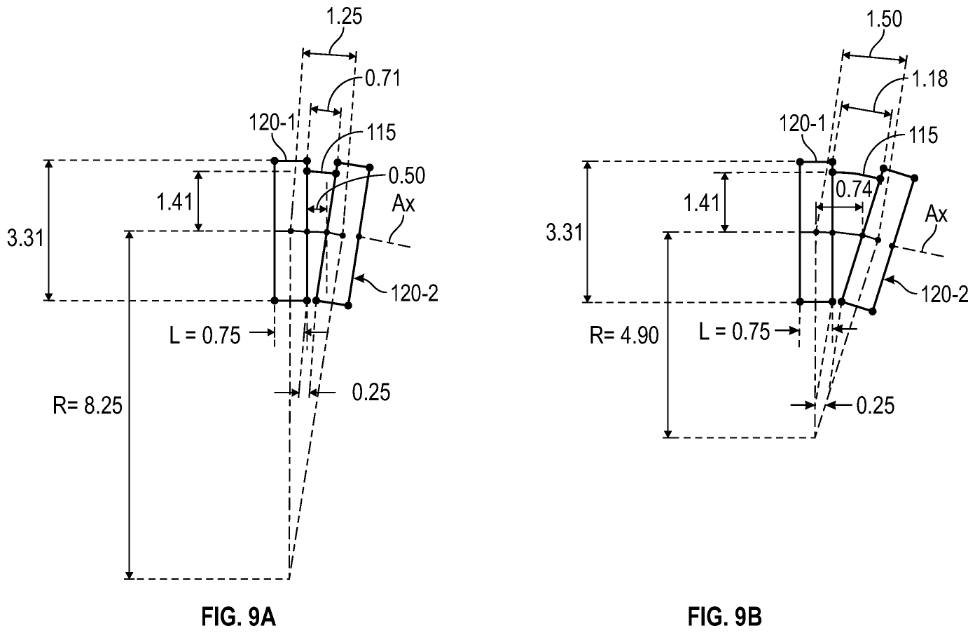

### FIG. 9A-9B: Reinforced Central Lumen Extrusion and Change of Gap distance between Guide Rings

According to at least one embodiment, the hoop strength and flexibility of a central lumen extrusion with reinforcing structure is further improved by adjusting (decreasing or increasing) the gap distance (D) between consecutive guide rings of one or more of the bending segments. According to one example embodiment, the shorter the gap distance between consecutive guide rings, the greater the increase in deflection resistance of the central lumen extrusion, and the opposite is also true. Therefore, a decreased gap distance between consecutive guide rings of at least one bending segment (in particular the distal end bending segment) will also minimize the incidence of the instrument getting caught on a guide ring during a procedure. However, careful consideration is necessary to achieve an appropriate amount (radius) of bending of the steerable instrument.

FIG. 9A and FIG. 9B illustrate an example embodiment of a reinforced central lumen extrusion with different gap distances between consecutive guide rings of a bending segment. In FIG. 9A and FIG. 9B, the central lumen extrusion is not shown for ease of illustration of a first guide ring 120-1 and a second guide ring 120-2 which are arranged consecutively along a lumen axis Ax. A drive wire 115 passes through a wire conduit of each guide ring 120-1 and guide ring 120-2. The drive wire 115 is operated to actuate (bend) the tubular body of the catheter sheath 100 by a pull or push movement so that the gap distance between consecutive guide-rings 120 decreases along the inner radius and increases on the outer radius, as explained elsewhere in this disclosure. The catheter sheath of the embodiment in FIG. 9A includes a smaller gap distance between the guide rings 120-1 and 120-2 than the embodiment in FIG. 9B. In this case, the catheter sheath according to the arrangement of FIG. 9A has a shorter length of the central lumen extrusion between the two consecutive guide rings 120-1 and 120-2. When an instrument or tool (e.g., a biopsy tool or camera) is inserted through the central lumen or tool channel 150 of the sheath in FIG. 9A, since the shorter central lumen extrusion in FIG. 9A has reduced deflection as compared to the embodiment in FIB. 9B, the tool does not catch the edge of a guide ring. Placing the guide rings closer together makes the central lumen extrusion effectively stiffer between the two guide rings. Thus more force will be required to deflect the central lumen extrusion enough to cause an instrument to catch on the guide ring. So, when the guide rings are placed at a smaller gap distance, the instrument or tool passing through the central lumen extrusion is more likely to follow the bend of the central lumen rather than to get stuck on the edge of the rings and try to poke out of the sheath through the space between the guide rings. From the foregoing, it can be appreciated that by reducing the gap distance between consecutive guide rings it is possible to improve navigation of instruments through the catheter sheath, and minimize damage to the sheath and/or tool.

### FIG. 10 and FIG. 11A-1113: Reinforced Outer Jacket

According to yet a further embodiment, both the outer jacket and the inner liner can be reinforced to provide enhanced hoop strength to the steerable sheath. FIG. 10 illustrates an example embodiment of a catheter sheath 100 having a reinforced central lumen extrusion 200 and an outer jacket 800. The catheter sheath 100 includes, arranged substantially concentric with a longitudinal axis Ax, the central lumen extrusion 200, a plurality of rings 120, and outer jacket 800, and a plurality of drive wires 115. The central lumen extrusion 200 defines a central lumen or tool channel 150 which is configured to pass medical tools and devices for treatment of a patient's anatomy. The plurality of guide rings 120 includes wire conduits 151 to pass the driving wires 115. The driving wires 115 receive an actuating force (push or pull force) to bend at least one bending segment of the steerable sheath 100. According to the various embodiments of the present disclosure, the catheter sheath 100 can bend more than 90 degrees (up to 180 degrees or more) with a minimum radius R of about 5.0 mm or less.

FIG. 11A shows a cross-sectional view of catheter sheath 100, as seen from a plane perpendicular to the lumen axis Ax. According to FIG. 11A, the central lumen extrusion 200 defines a central lumen or tool channel 150 enclosed by a tubular wall made of an inner layer 210a, a reinforcing structure 220, and an outer layer 210b. A guide ring 120 is bonded to the outer surface (outer layer 210b) of the extrusion 200. The guide ring 120 includes a plurality of wire conduits 151 configured to pass at least one drive wire 115 through one or more wire conduit. Some wire conduits 151 can be unused or can be used to pass other types of wires. The outer jacket 800 encloses the guide rings 120. In FIG. 11A, the reinforcing structure 220 of the central lumen extrusion 200 is offset towards the inner surface such that the inner layer 210a is thinner than the outer layer 210b.

FIG. 11B shows a cross-sectional view of catheter sheath 100 similar to that shown in FIG. 11A. According to FIG. 11B, the outer jacket 800 is also reinforced by a reinforcing structure 820 which is similar to the reinforcing structure 220. According to at least one embodiment, the outer jacket 800 includes an inner layer 810a, a coiled wire reinforcing structure 820, and an outer layer 810b. In this case, the coil in the reinforced central lumen extrusion 200 (inner liner) could be wound in the opposite direction to the coil in the reinforced outer jacket 800. For example, the coil in the reinforcing structure 220 is wound in a clockwise (CW) direction, while the coil in the reinforcing structure 820 is wound in a counter-clockwise (CCW) direction, as shown by opposing arrows. In addition, similar to the central lumen extrusion 200, the outer jacket 800 can have the reinforcing structure 820 offset towards the inner or outer surface. In the example shown by FIG. 11B, the reinforcing structure 820 of the outer jacket is offset towards the outer surface to provide more resistance to outside pressuring forces. The combination of opposite wounded coils in the central lumen extrusion and the outer jacket would improve torsional stiffness while maintaining sufficient hoop strength and flexibility of the sheath.

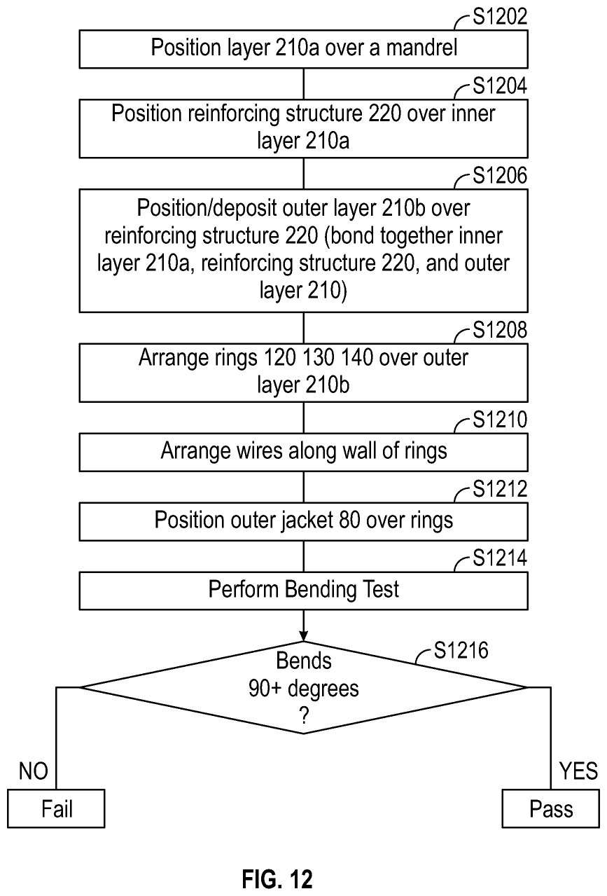

### FIG. 12: Exemplary Manufacturing Process

FIG. 12 illustrates an overall process of manufacturing a catheter sheath in accordance with one embodiment of the present disclosure. For example, the process of FIG. 12 represents possible steps for crating a steerable catheter sheath for a snake-like continuum robot as illustrated in FIG. 2A. The steps of FIG. 12 can be modified (added or reduced) depending on the type of application for which the catheter sheath is created. In an exemplary manufacturing process of a catheter sheath, first a reinforced central lumen extrusion is formed, second a plurality of rings are arranged over the central lumen extrusion, third an outer jacket is arranged over the plurality of rings. The rings can have thru-holes or secondary lumens formed and arranged to surround the central lumen. The rings can be pressure fit, bonded, welded, or attached in any other manner to the outer surface of the central lumen extrusion and/or to the inner surface of the outer jacket. To ensure that the catheter sheath meets minimum requirements, a final step of the process is to perform a bending test. These steps can be performed any type of manufacturing process as known to persons having ordinary skill in the art of medical devices.

In one example, at step S1202 a thin inner layer 210a is first loaded onto a mandrel (not shown). At step S1204, the reinforcing structure 220 is positioned over the inner layer 210a. As mentioned elsewhere, the reinforcing structure can include one or more of a braid structure, a coil structure, and a laser-cut tube structure, or combinations thereof. At step S1206, an outer layer 210b is positioned over the reinforcing structure 220. At this point, any known process takes place to bond together the inner layer 210a, reinforcing structure 220, and the outer layer 210b. Depending on the desired catheter structure, the inner layer 210a can be thinner than the outer layer 210b, so that the reinforcing structure 220 is offset towards the inner surface of the central lumen extrusion. Alternatively, the inner layer 210a can be thicker than the outer layer 210b, so that the reinforcing structure 220 is offset towards the outer surface of the central lumen extrusion. Furthermore, the inner layer 210a can have a higher or lower durometer than the outer layer 210b and vice versa. At step S1204, any of the reinforcing structure including the braid structure, the coil structure, the laser-cut structure or combinations thereof can be arranged along the length of the inner layer 210a. At step S1206, bonding the inner layer 210a and outer layer 210b with the reinforcing structure therebetween can be done by any known process including one or more of pressure fitting, welding (e.g., ultrasonic or laser), bonding with adhesive materials, bonding with thermal processes (for example reflowing, curing with UV energy, or heat shrinking the outer layer over the reinforcing structure). Moreover bonding can be done continuously along the entire length of the central lumen extrusion, or only at selected sections where the reinforcing structure is applied.

In some embodiments, the inner liner 210 can be an off-the-shelf commercially available reinforced tube. In such embodiment, the inner liner can be a braid-reinforced polymer tube such as a braided 40D Pebax tube. In this case, the steps S1202 to S1206 may be optional to add an extra layer of reinforcing structure only at certain locations of the central lumen extrusion. Alternatively, with an off-the-shelf reinforced inner liner, the process can start at step S1208.

At step S1208, a plurality of first rings 120, a plurality of second rings 130, and a plurality of third rings 140 (as shown in FIG. 2B) are arranged over the outer layer 210b of the central lumen extrusion. As shown in FIG. 2C and FIG. 2D, the rings can have secondary lumens or thru-holes 151-159, but at least some of the rings may have no thru-holes. Here, when arranging the rings over the central lumen extrusion, the rings can be pressure fit over the outer surface (outer layer 210b) of the inner liner 210. Optionally, at step S1208, the rings can be welded or bonded or otherwise affixed to the outer surface of the outer layer 210b. Attaching the rings to the central lumen extrusion can be done by any known process including one or more of pressure fitting, welding (e.g., ultrasonic or laser welding), bonding with adhesive materials, bonding with a thermal processes (for example reflowing, curing with UV energy, etc.). As understood by those skilled in the art, laser welding can be considered as part of bonding with a thermal process. In some embodiments, a special primer and adhesive material specifically designed to provide greater bonding strength with polymer materials such as PTU or PTE can be used. One example of a priming material to promote bonding to such materials is Loctite® SF 770 (known as Loctite 770) and commercially available from the Henkel Corporation. The elastomeric or polymer layers of the catheter sheath, which does not usually bond well with conventional adhesives, can be coated or jacketed with epoxy or other material that allows for better bonding.

At step S1210, one or more wires can be arranged in the thru-holes of the rings along the wall of the rings. In some embodiments, wires can be arranged along slots (e.g., slot 131 in FIG. 2D) formed on the outer surfaces of the rings. In a catheter sheath for a snake-like continuum robot, wires can include one or more of driving wires (control wires to actuate one or more of the bending segments), support wires that function as tendons or backbone (not actuated wires) for the robot, or cable wires (electrical cables made of one or more metal strands that transmit electrical signals). In addition, the thru-holes or slots formed on the rings may be used to arrange therein elongated sensors such as electromagnetic (EM) sensors, optical fibers, radiopaque markers, and other similar components.

At step S1212, an outer jacket 80 is arranged over the entire structure covering the plurality of rings 120, 130, 140 of the steerable distal section 3, the central lumen extrusion 200, and the non-steerable proximal section 4. At this step, the rings can be additionally welded or bonded or otherwise affixed to the inner surface of the outer jacket 80.

At step S1214, a bending test is performed to ensure that the newly formed catheter sheath meets the necessary requirements. For example, at step S1213, the bending test confirms if the catheter sheath bends at least 90 degrees (90+ degrees) without causing a tool or instrument to get stacked. To that end, several tests can be performed such as bending the sheath at different radiuses of curvature, and passing a tool or instrument through the central lumen numerous times to assess whether the sheath can pass or fail such strenuous use.

Any or all of the previous embodiments could be combined to incrementally improve the performance of the catheter.

The foregoing embodiments are directed to a single inventive concept of a steerable sheath having a reinforced central lumen with enhanced loop strength and increased flexibility. The steerable sheath of a snake-like continuum robot is configured to guide a medical instrument through the reinforced central lumen by manipulating (kinematically actuating) one or more bending sections of the sheath. According to the various embodiments, the reinforced central lumen extrusion includes one or more of the following features and provides one or more of the following advantages.

Main Feature: A flexible catheter sheath including a central lumen extrusion, guide rings, outer jacket. The guide rings are bonded to the central lumen extrusion at a predetermined distance from each other. The outer jacket is outside of the guide rings. The central lumen extrusion is a tubular body that includes a braid or coil or laser-cut tube structure in the wall of the tubular body.

Dependent Feature 1: The central lumen extrusion has: an inner layer, which is from an inner surface to the braid or coil or laser-cut structure, and an outer layer, which is from the braid or coil or laser-cut structure to the outer surface. The inner layer is made of a material more lubricious than the material of the outer layer.

Dependent Feature 2: The same as Dependent Feature 1, wherein the outer layer is made of thermoplastic elastomer. Dependent Feature 2a: The same as Dependent Feature 1, wherein the outer layer is made of thermoplastic elastomer mixed with carbon black.

Dependent Feature 3: The same as Dependent Feature 1, wherein the wall thickness of the inner layer is larger than the wall thickness of the outer layer.

Dependent Feature 4: The same as Dependent Feature 1, wherein the wall thickness of the inner layer is smaller than the wall thickness of the outer layer.

Dependent Feature 5: The same as Dependent Feature 1, wherein the hardness durometer of the inner layer is lower than the hardness durometer of the outer layer.

Dependent Feature 6: The same as Dependent Feature 1, wherein hardness durometer of the inner layer is higher than the hardness durometer of the outer layer.

Dependent Feature 7: The central lumen extrusion has: a first coil structure in the wall of tubular body; the outer jacket has a second coil structure in the wall of the outer jacket. The coiling of the first coil and second coil is done by winding a metallic wire over an inner surface (inner liner) and applying medical-grade thermoplastic elastomer over the coiled wire. The winding direction of the first and second coil structures are opposite to each other.

Dependent Feature 8: The central lumen extrusion is a reinforced flexible tubular body that has plural layers between an inner surface and an outer surface. The plural layers include: an inner layer, which is from the inner surface to the braid or coil or laser-cut structure; an outer layer, which is from the braid or coil or laser-cut structure to the outer surface. The outer layer includes carbon black while the inner layer does not include carbon black.

Dependent Feature 9: A catheter sheath comprising the central lumen extrusion according to any of Features 1-8, further comprising a plurality of rings arranged on the outer surface of the central lumen extrusion.

Dependent Feature 10: The same as Dependent Feature 9, wherein the rings are made of transparent/translucent material, and wherein the rings are bonded to the outer surface of the central lumen extrusion by one or more of pressure fitting, welding (laser welding or ultrasonic welding), bonding with adhesive material, bonding with thermal process (for example, reflowing or UV curing).

Advantages of a adding a reinforcing structure: Braid Reinforcement provides: Increased torsional stiffness; increased hoop strength; manufactured in continuous length (lower cost); Coil Reinforcement provides: Lower wall thickness of reinforcement; increased hoop strength; Laser Cut Tube Reinforcement provides: More resistance to compression; increased hoop strength; increased torsional stiffness;

Advantage of using multiple materials/durometers for layers of inner liner: Greater lubricity on ID side from braid/coil than the material on OD side to help for tool to pass though the channel;

Different materials/durometers for inner liner as compared to outer jacket: Use of thermoplastic elastomer optionally adding carbon black on OD side from braid/coil to allow bonding the guide rings with reflow/laser welding process. Use lower durometer materials for outer jacket to provide improved flexibility for navigation (insertion and withdrawal);

In one embodiment, the reinforcement structure (braid, coil, or laser-cut tube) is offset toward the inner side in the wall thickness. This provides improved compatibility with downstream processes, which includes bonding the guide rings to the outer surface of inner liner with either by reflow or laser welding process. The offset reinforcement structure also provides increased hoop strength;

In one embodiment, the reinforcement structure (braid, coil, or laser-cut tube) is offset toward the outer side in the wall thickness. This provides reduce risk to expose reinforcement structure to the tool channel, and improves lifetime of device for multiple tool usage;

In one embodiment, the inner layer of central lumen extrusion is made of low durometer material, and outer layer is made of high durometer material. This is advantageous for maintaining good bending flexibility;

In one embodiment, the inner layer of central lumen extrusion is made of high durometer material, and the outer layer is made of low durometer material, and optionally with added lubricious coating or lubricious additive. This provides slippery and rigid inner surface to promote smooth tool handling;

In one embodiment, coil reinforcement is added to both the Inner Liner and Outer jacket. This provides improved torsional stiffness, increased hoop strength, and helps maintain bending flexibility in central lumen extrusion.

In one embodiment, only the outer layer of inner liner includes carbon black additive. This improves compatibility with downstream manufacturing process, which is bonding the guide rings to the outer surface of the inner liner with laser welding. Due to outer layer including carbon black additive, only the outer layer becomes black color and can absorb laser light and be heated selectively. Since inner layer of inner liner is not heated by the laser welding, it is possible to reduce a risk to melt the inner surface or change the smoothness of the lumen;

Other advantages include Increased Lubricity: Lower insertion forces; material cost increase is minimal; Decreased Gap between guide rings: Reduced chance for instrument to catch on guide rings; Reinforced OD of inner liner with annulus structure formed on central lumen extrusion between guide rings: Increases hoop strength of the central lumen;

Chamfered, beveled or curved inner edges of Guide Rings and/or grooved outer diameter of the central lumen extrusion: Decreased chance for instrument to catch on a ring during insertion.

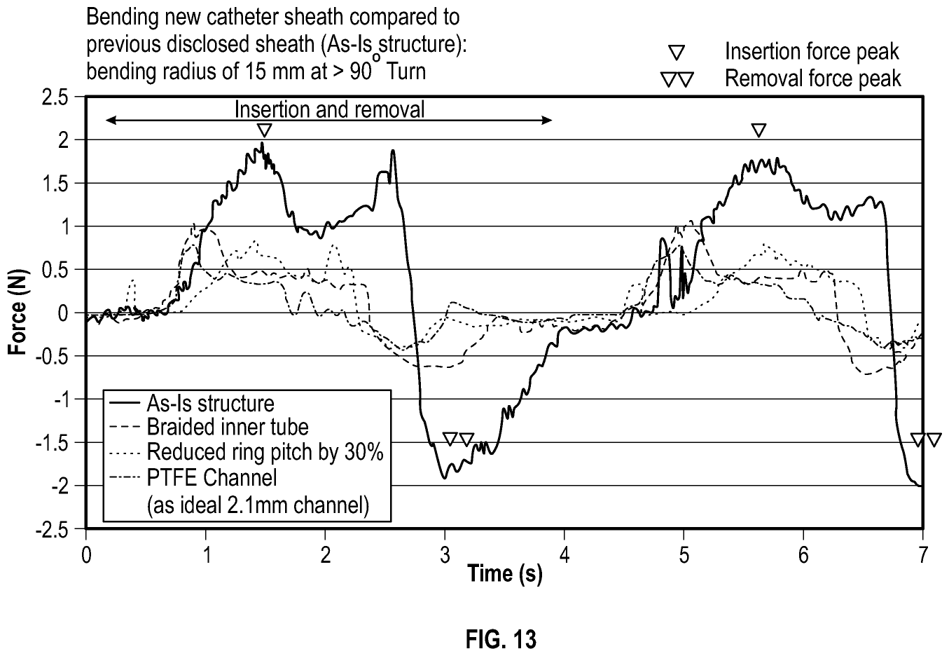

### FIG. 13: Experimental Results

Experiments were run to assess, in a variety of ways, how to create an improved bendable body for a steerable medical instrument with the foregoing features. Tests were performed to simulate an environment where a bendable medical instrument accepts a variety of tools into the tool channel without becoming stuck, particularly when the medical instrument is situated in vivo and subjected to a tortious environment, and is needed to undergo one or more tight bends (e.g., bends of more than 90 degrees at a relatively small radius).

Experiments were performed using a catheter sheath design shown in any of FIG. 3, FIG. 4, or FIG. 5 a bendable body having a single central lumen extrusion with a tool channel of 0.089 inches ID, 0.099 inches OD, was made of Pebax® and having an average hardness durometer of 35D Shore. In one embodiment, the central lumen extrusion had a PTFE channel with an ideal smooth surface machined from a PTFE block similar to a previous catheter structure described by the applicant in publication WO/2020/092097. However, in the present disclosure, the central lumen extrusion is reinforced by a braided reinforcing structure. In other embodiment, at least the inner surface (inner layer) of the central lumen extrusion is made of a permeable material, such as expanded polytetrafluoroethylene (ePTFE). Since the inner layer is microporous, the inner surface can be added a highly lubricious material to improve the passing of medical tools without getting stuck on the rings. In some embodiments, the inner liner has a lubricious additive such that the inner surface in lubricated. Such additives include, but are not limited to, Moblize, Pebaslix, and Propell.

One embodiment is directed to a steerable catheter with reduced pitch rings: The reinforced central lumen extrusion was used to build a steerable catheter having a structure similar to that shown in FIG. 2A, FIG. 9A and FIG. 9B. The catheter with reduced pitch rings has a reduced ring pitch (the ring pitch is reduced by as much as 30% in comparison to catheters previously disclosed by applicant).

Experimental catheter prototypes according to the above-mentioned embodiments were tested and compared to a previously disclosed “as is” snake-like catheter. In the “as is” tested snake catheter, the rings were 1 mm wide with a spacing gap of 1 mm. The new catheter with reduced pitch rings was built with 0.75 mm rings and a 0.75 mm gap distance between consecutive rings. In this embodiment, the catheter having rings 0.75 mm and 0.75 mm spacing gap is reduced from the 1 mm wide gap. The results showed significant improvement in insertion performance while maintaining minimum bend radius performance.

FIG. 13 shows a graph with experimental results of bending the new catheter sheath prototypes as compared to the previously disclosed catheter sheath (“As-Is” structure). These experiments were based on a bending radius of 15 mm with a bending curvature of at least 90 degrees to about 180 degrees.

From FIG. 13, it can be appreciated that adding a braided reinforcing structure to the inner layer (dashed line), reducing the ring pitch by about 30% (dotted line), and making the inner surface (tool channel) of a permeable material (ePTFE) and adding lubricious additives such as Moblize, Pebaslix, and Propell to the inner layer can reduce the force necessary for insertion and removal of the catheter through tortuous paths.

According to several experiments, reducing the ring width and the gap distance in a one to one ratio provided significantly better results than reducing the width of the ring or gap distance alone. Keeping a 1:1 ratio of ring width to gap distance outperforms significantly from a minimum bend radius perspective. For example, reducing the gap distance alone (for example, if you keep a 1 mm ring width and reduce only the gap between rings to 0.5 mm, you end up having a minimum bend radius of 10 mm vs the 5 mm the 0.75×0.75 provides.

With less ring surface area to bond/laser weld to, such as defined in applicants previously filed patent application publication US 2021/0259790, herein incorporated by reference in its entirety. In accordance with this prior publication, some options are to laser weld the rings to the outer cover (outer jacket) instead of the central lumen extrusion as the surface area is larger and the surface is right there (easier to reach).

In terms of tool insertion performance, a catheter having 1.0 mm ring width and 0.5 mm gap distance between rings performed best. But in terms of bending radius, this catheter had the biggest bend angle. On the other hand the catheter with rings of 0.75 mm with and 0.75 mm gap distance provided lower tool insertion performance but had significantly less bend radius, making it a preferred embodiment for some applications. A catheter having ring width of 0.75 mm and 0.5 mm gap distance also performed well.

The braided inner tube may be, for example an off the shelf braid reinforced polymer tube such as a braided 40D Pebax®. This braided inner tube may be attached to the ring structure and to the outer tube by any known method. For example, laser welding may be used. In some embodiments the laser welding may be biased to the more proximal and/or more distal end of the tool channel. A thicker structure may be provided at these region(s) to improve the laser weld. The braided inner tube may be selected to retain the flexibility of the snake robot. Additives (e.g., lubricious materials) may be added to the inner diameter or to both the inner and outer diameters. The braided inner tube may be made with separate materials inside and outside of the braid. In one embodiment, the inner layer includes a lubricious additive and the outer layer does not.

The braided inner tube may be, for example an off the shelf braid reinforced polymer tube such as a braided 40D pebax. This braided inner tube may be attached to the ring structure and to the outer tube by any known method. For example, laser welding may be used. In some embodiments the laser welding may be biased to the more proximal and/or more distal end of the tool channel. A thicker structure may be provided at these region(s) to improve the laser weld. The braided inner tube may be selected to retain the flexibility of the snake robot. Additives (e.g., lubricious materials) may be added to the inner diameter or to both the inner and outer diameters. The braided inner tube may be made with separate materials inside and outside of the braid. In one embodiment, the inner portion includes a lubricious additive and the outer portion does not.

Catheter with inner tube with low Poisson's Ratio: In some embodiments, the inner tube or liner has a Poisson's Ratio less than a defined amount. When the Poisson's Ratio is lower than this amount, the wrinkles of the inner liner at an acute bend radius are eliminated. Also, since the liner would have little to no state of stress, there is no force to counteract thus is able to hold the catheter pose more easily.

### Other Embodiments and Modifications

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that changes may be made in details, particularly in matters of shape, size, and arrangement of components or steps without departing from the scope of the invention. Therefore, the scope of the following claims is to be accorded the broadest reasonable interpretation so as to encompass all such modifications and equivalent structures and functions.

What is claimed is:

1. A catheter sheath extending lengthwise from a proximal end to a distal end along a sheath axis, the catheter sheath comprising:
a central lumen extrusion having multiple layers including an inner layer defining a central lumen, a reinforcing structure surrounding the inner layer, and an outer layer surrounding the reinforcing structure in this order substantially concentric with the sheath axis; and
a plurality of rings arranged on the outer layer of the central lumen extrusion, wherein the plurality of rings are arranged at a predetermined distance from each other in a direction from the distal end towards the proximal end; and
wherein the reinforcing structure of the central lumen extrusion includes one or more of a braid structure, a coil structure, and a laser-cut tube structure embedded between the inner layer and the outer layer, and
wherein the central lumen extrusion is bonded and/or pressure fit to one or more of the plurality of rings.

2. The catheter sheath according to claim 1,
wherein the reinforcing structure is offset towards the inner surface or towards the outer surface of the central lumen extrusion such that a thickness of the inner layer is different from a thickness of the outer layer.

3. The catheter sheath according to claim 1,
wherein each of the rings has an inner surface and an outer surface made of thermoplastic polymer,
wherein the outer layer of the central lumen extrusion is made of thermoplastic polymer, and
wherein each of the rings is attached to the outer layer of the central lumen extrusion by pressure fitting or bonding with an adhesive or bonding with a heating process such that the inner surface of each ring is fixedly attached to the outer layer of the central lumen extrusion.

4. The catheter sheath according to claim 1,
wherein the braid structure includes braided polymeric fibers and/or braided metallic strands arranged between the inner layer and the outer layer;
wherein the coil structure includes a coiled metallic wire and/or a coiled polymeric filament arranged between the inner layer and the outer layer; and
wherein the laser-cut tube structure includes a metallic tube and/or a polymer-based tube with a slot cut patter, and the laser-cut tube structure is arranged between the inner layer and the outer layer.

5. The catheter sheath according to claim 1, further comprising:
an outer jacket enclosing the plurality of rings and at least part of the central lumen extrusion,
wherein each of the rings has an inner circumference in contact with the outer layer of the central lumen extrusion and an outer circumference enclosed by the outer jacket.

6. The catheter sheath according to claim 1,
wherein each of the rings has an inner surface in contact with the outer layer of the central lumen extrusion, and
wherein two or more of the rings have chamfered or beveled or rounded edges on the inner surface thereof such that when the central lumen extrusion is bent, the chamfered or beveled or rounded edges of the rings minimize pressure of the rings against the outer layer of the central lumen extrusion.

7. The catheter sheath according to claim 1,
wherein the outer layer of the central lumen extrusion is made of thermoplastic polymer combined with carbon black, and
wherein the rings are made of transparent or translucent polymer material.

8. The catheter sheath according to claim 1,
wherein the inner layer of the central lumen extrusion is made of elastomeric polymer combined or coated with a lubricious additive.

9. The catheter sheath according to claim 1,
wherein each of the rings has a length in the lengthwise direction, and
wherein the length of each of the rings is equal to or smaller than the predetermined distance at which the rings are arranged.

10. The catheter sheath according to claim 9, wherein a ratio of the length of each ring to the predetermined distance at which the rings are arranged is in a range of 3 to 0.3, or in a range of 2 to 0.5, or in a range of 1.5 to 1.

11. The catheter sheath according to claim 9,
wherein the length of each ring and the predetermined distance at which the rings are arranged are respectively 1 mm and 0.5 mm, or 0.75 mm and 0.75 mm, or 0.75 mm and 0.5 mm.

12. The catheter sheath according to claim 1,
wherein the catheter sheath includes a non-steerable section and a steerable section in order from the proximal end to the distal end,
wherein the steerable section corresponds to a part of the central lumen extrusion where the rings are arranged,
wherein the non-steerable section corresponds to a part of the central lumen extrusion where the rings are not arranged,
wherein the non-steerable section is located proximal to the rings, and the non-steerable section has substantially a same diameter as the diameter of the rings.

13. The catheter sheath according to claim 5,
wherein the outer jacket includes an inner layer, a reinforcing structure, and an outer layer arranged in this order substantially concentric with the sheath axis,
wherein the reinforcing structure of the outer jacket is formed by a coiled metallic wire and/or a coiled polymeric wire, and
wherein the coiled metallic wire and/or the coiled polymeric wire of the central lumen extrusion is coiled in an opposite direction to the coiled metallic wire and/or coiled polymeric wire of the outer jacket.

14. The catheter sheath according to claim 1,
wherein the central lumen extrusion includes a first polymer layer that forms the inner surface and a second polymer layer that forms the outer surface, and
wherein the reinforcing structure is offset towards the outer surface of the central lumen extrusion such that a thickness of the first polymer layer is greater than a thickness of the second polymer layer.

15. The catheter sheath according to claim 1,
wherein the central lumen extrusion includes a first polymer layer that forms the inner surface and a second polymer layer that forms the outer surface, and
wherein the reinforcing structure is offset towards the inner surface of the central lumen extrusion such that a thickness of the first polymer layer is smaller than a thickness of the second polymer layer.

16. The catheter sheath according to claim 5,
wherein the central lumen extrusion includes a first polymer layer that forms the inner layer and a second polymer layer that forms the outer layer,
wherein the outer jacket includes an inner layer, a reinforcing structure, and an outer layer arranged in this order substantially concentric with the sheath axis,
wherein the reinforcing structure of the outer jacket is offset towards the inner surface or towards the outer surface thereof such that a thickness of the inner layer is different from the thickness of the outer layer of the outer jacket.

17. The catheter sheath according to claim 1,
wherein the central lumen extrusion includes a first layer of thermoplastic polyurethane (TPU) that forms the inner surface and a second layer of thermoplastic elastomer (TPE) that forms the outer surface, and
wherein only the second layer of TPE includes carbon black additive material and the first polymer layer does not include carbon black additive material such that outer surface of the central lumen extrusion is configured to absorb laser welding heath at a greater rate than the inner surface.

18. The catheter sheath according to claim 1,
wherein the rings include guide rings comprising wire-guiding conduits arranged substantially parallel to and equidistant from the sheath axis;
wherein at least one wire-guiding conduit in each guide ring includes at least one control wire slideably arranged along the length of the central lumen extrusion, a distal end of the at least one control wire being attached to an steerable segment of the steerable section and a proximal end of the at least one control wire configured to be mechanically connected to an actuator unit.

19. The catheter sheath according to claim 1, further comprising:
one or more reinforcing rings arranged on the outer surface of the central lumen extrusion in a gap between one or more pairs of the rings arranged at the predetermined distance,
wherein each reinforcing rings has a length smaller the predetermined distance, and
wherein the diameter of each of the reinforcing rings is smaller than the diameter of each of the rings arranged at the predetermined distance.

20. The catheter sheath according to claim 1,
wherein the wherein the catheter sheath includes a steerable section which corresponds to a part of the central lumen extrusion where the rings are arranged,
wherein the steerable section includes a plurality of bending segments, and
wherein the central lumen extrusion corresponding to each bending segment is reinforced by a different reinforcing structure selected from the braid structure, the coil structure, and the laser-cut tube structure embedded between the inner layer and the outer layer.
